(19) United States
(12) Patent Application Publication
(10) Pub. No.: US 2023/0157283 A1
(43) Pub. Date: May 25, 2023

(54) COMPOSITIONS AND METHODS FOR INHIBITING A FUNGAL PATHOGEN

(71) Applicants: Tu Biomics, Inc., Reno, NV (US); Board of Regents of the Nevada System of Higher Education, on behalf of the Desert Research Institut, Reno, NV (US)

(72) Inventors: John Albert ARNONE, III, Reno, NV (US); Richard Louis JASONI, Reno, NV (US); David Charles VUONO, Reno, NV (US); Brian SPEICHER, Reno, NV (US)

(73) Assignees: Tu Biomics, Inc., Reno, NV (US); Board of Regents of the Nevada System of Higher Education, on behalf of the Desert Research Institut, Reno, NV (US)

(21) Appl. No.: 17/908,516

(22) PCT Filed: Mar. 2, 2021
(86) PCT No.: PCT/US2021/020431

§ 371 Date: Aug. 31, 2022

Related U.S. Application Data
(60) Provisional application No. 62/984,956, filed on Mar. 4, 2020.
(60) Provisional application No. 62/992,364, filed on Mar. 20, 2020.
(60) Provisional application No. 63/110,517, filed on Nov. 6, 2020.

(51) Int. Cl. A01N 37/36; A01P 3/00

(52) U.S. Cl. CPC A01N 37/36; A01P 3/00

(57) ABSTRACT

Provided are methods and compositions for controlling growth of fungi.

### CROSS-REFERENCE TO RELATED APPLICATION

This application claims the benefit of the following U.S. Provisional Applications No.: 62/984,956, filed Mar. 4, 2020; 62/992,364, filed Mar. 20, 2020; and 63/110,517, filed Nov. 6, 2020; the entire contents of which are incorporated herein by reference.

### BACKGROUND OF THE INVENTION

At the scale of industrial agriculture, where disease pressure has an economically meaningful impact globally, a continually narrowing selection of agrochemicals continues to be applied with increasing examples of pathogen resistance, and with deleterious effects on soil health, the environment, and human health. Efforts have been made to develop more sustainable ways of controlling agricultural diseases; however, the methods are difficult to implement or yield unpredictable results. For example, anaerobic soil disinfestation (ASD) is characterized by inconsistent results and is prohibitive in scale and expense, thereby limiting opportunities for optimization or application in commercial scale agriculture.

Therefore, there remains a need for the development of safe and environmentally friendly compositions and methods for effectively and economically promoting plant health and controlling and/or mitigating the growth of pathogens that have a deleterious effect in global food production and plant health.

### SUMMARY OF THE INVENTION

As described below, the present invention features compositions comprising lactate and acetate and methods of using such compositions for inhibiting the growth of fungal pathogens (e.g., Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahlia).In one aspect, the invention features a composition containing levorotatory lactate (L-Lactate) and acetate, where the composition is substantially free of dextrorotatory lactate (D-Lactate).

In one aspect, the invention features a method for preparing soil and/or protecting plant surfaces for/while growing a food crop plant, ornamental plant, tree, or turf The method involves contacting the soil and/or plant surfaces with a composition containing L-lactate and acetate, thereby preparing the soil and/or protecting the plant.

In any of the above aspects, the amount of each of L-lactate and acetate is effective to inhibit the growth or survival of a fungal pathogen contacted with the composition. In any of the above embodiments, the composition further contains a carrier.

In any of the above aspects, the composition contains from about 50 ppm to about 1,600 ppm L-lactate and from about 50 ppm to about 1,600 ppm acetate. In any of the above aspects, the composition contains at least about 400 ppm L-lactate and at least about 400 ppm acetate. In any of the above aspects, the composition contains at least about 800 ppm L-lactate and at least about 800 ppm acetate.

In one aspect, the invention features a method for reducing or eliminating growth of a fungus. The method involves contacting the fungus with L-lactate and acetate, thereby reducing or eliminating growth of the fungus.

In one aspect, the invention features a method for inhibiting fungal disease in a food crop plant, ornamental plant, tree, or turf. The method involves contacting the fungus with L-lactate and acetate, thereby inhibiting the fungal disease.

In one aspect, the invention features a method for inhibiting white rot in an Allium plant and/or an Allium growth medium. The method involves contacting the Allium plant and/or the growth medium with L-lactate and acetate, thereby inhibiting white rot in the Allium plant and/or the Allium growth medium.

In one aspect, the invention features a method for inhibiting gray mold in a plant or growth medium. The method involves contacting the plant or growth medium with L-lactate and acetate, thereby inhibiting gray mold.

In one aspect, the invention features a plant growth medium containing L-lactate and acetate, where the plant growth medium is substantially free of dextrorotatory lactate (D-Lactate).

In any of the above aspects, the plant belongs to the Allium genus. In any of the above aspects, the plant is Allium sativum, Allium cepa, Allium chinense, Allium stipitatum, Allium schoenoprasum, Allium tuberosum, Allium fistulosum, or Allium ampeloprasum. In any of the above aspects, the fungus belongs to a genus selected from one or more of Botrytis, Collelotrichum, Fusarium, Macrophomina, Phytophthora, Pythium, Rhizoctonia, Sclerotinia, Sclerotiniaceae, Sclerotium, and Verticillium. In any of the above aspects, the fungus is Sclerotium cepivorum. In any of the above aspects, the fungus is Phytophthora cactorum. In any of the above aspects, the fungus is Botrytis cinerea. In any of the above aspects, the fungus is Colletotrichum acutatum. In any of the above aspects, the fungus is Fusarium oxysporum f. sp. fragariae. In any of the above aspects, the fungus is Macrophomina phaseolina. In any of the above aspects, the fungus is Pythium uncinulatum. In any of the above aspects, the fungus is Rhizoctonia solani. In any of the above aspects, the fungus is Sclerotinia minor. In any of the above aspects, the fungus is Sclerotium cepivorum. In any of the above aspects, the fungus is Sclerotinia sclerotiorum. In any of the above aspects, the fungus is Verticillium dahlia.

In any of the above aspects, the fungus is present in soil or another growth medium.

In any of the above aspects, the fungus is present above ground, on a plant.

In any of the above aspects, the method reduces a fungal growth rate in soil.

In any of the above aspects, the method reduces a fungal growth rate on a plant.

In any of the above aspects, contacting involves use of sprinklers, spraying, dripping, or drenching. In any of the above aspects, fungus is present in soil and is contacted with L-lactate and acetate by soil drenching. In any of the above aspects, fungus is present on a plant and is contacted with L-lactate and acetate from a spray bottle, a sprayer, a nozzle, a sprinkler, or a drip line.

In any of the above aspects, after the contacting, the concentration of L-lactate in the soil or growth medium and/or on the plant is from about 40 ppm to about 5000 ppm and the concentration of acetate in the soil or growth medium is from about 50 ppm to about 5000 ppm

In any of the above aspects, after the contacting, the plant surface or soil contains L-lactate and acetate at a weight ratio (lactate:acetate) of from about 1:6 to about 6:1.

In any of the above aspects, the growth medium is substantially free of viable pathogenic fungi.

In any of the above aspects, the growth medium is a liquid or solid.

In any of the above aspects, the growth medium is potting soil.

The invention provides compositions and methods for inhibiting the growth of a fungal pathogen (e.g., Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahliae). Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “L-lactate” or “L-lactic acid” is meant a compound having the chemical formula C₃H₆O₃, corresponding to CAS Number 79-33-4, having the structure

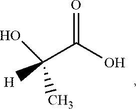

, and agronomically acceptable salts thereof. The salt can be a lithium, sodium, or potassium salt.

By “D-lactate” or “D-lactic acid” is meant a compound having the chemical formula C₃H₆O₃, corresponding to CAS Number 10326-41-7, and having the structure

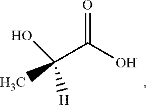

, and agronomically acceptable salts thereof. The salt can be a lithium, sodium, or potassium salt.

The term “lacate” can refer to D-lactate, L-lactate, or mixtures thereof.

By “acetate” or “acetic acid” is meant a compound having the formula C₂H₄O₂, corresponding to CAS Number 64-19-7, and having the structure

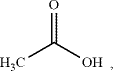

and agronomically acceptable salts thereof. The salt can be a lithium, sodium, or potassium salt.

By “agent” is meant any small molecule chemical compound. The small molecule chemical compound can be an organic acid (e.g., lactic acid and/or acetic acid).

By “agricultural field” is meant an area of land under cultivation or to be used for cultivating crops.

By “ameliorate” is meant decrease, suppress, attenuate, diminish, arrest, or stabilize the development or progression of a disease. In some embodiments, the disease is associated with a fungal pathogen (e.g., Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahliae). In some embodiments, the disease is gray mold or white rot.

By “carrier” is meant a substance that functions to facilitate the application of a composition to a plant or soil.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “concentrate” is meant a composition containing a high concentration of components because of lack of a solvent. A concentrate can be referred to as 2×, 3×, 4×, 5×, etc. depending on how many-fold the concentrate must be diluted using a solvent (e.g, water) to obtain a target, or working, concentration of the composition components. The concentrate can be a 1.5×, 2×, 3×, 4×, 5×, 10×, 15×, 20×, 25×, 50×, 75×, 100×, 150×, 200×, 250×, 300×, 500×, 750×, or 1,000× concentrate.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected.

By “disease” is meant any condition or disorder that damages or interferes with soil or plant function. The normal function of a soil includes the ability to sustain growth of a disease-free plant therein. The disease can be caused by a plant or soil pathogen (e.g., fungi). In some embodiments, the plant disease is white rot or gray mold. Pathogenic fungi include, for example, Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, and Verticillium dahliae. By “effective amount” is meant the amount of an agent required to ameliorate the symptoms of a disease relative to an untreated soil or plant. The effective amount of active compound(s) used to practice the present invention for treatment or prevention of a fungal disease (e.g., white rot, gray mold) varies depending upon the manner of administration and the plant and/or soil being treated. Such amount is referred to as an “effective” amount. In some embodiments, an effective amount is the amount required to inhibit fungal growth or to kill the fungus.

By “growth medium” is meant a solid, liquid, or semi-solid that functions to support growth of a plant. In some embodiments, the growth medium is a soil. In some embodiments, the growth medium contains soil, bark, clay (e.g., calcined clays), coir pith, green compost, peat (e.g., black peat or white peat), perlite, rice hulls, sand, grit, wood fibers, peat, vermiculite, leaf mold, sawdust, bagasse, expanded polystyrene, urea formaldehydes, or a combination thereof. In some embodiments, the growth medium is a hydroponic growth medium.

By “mitigate” is meant alleviating or reducing a pathogen or harmful effects thereof. As used herein “eliminate” refers to eradication of a pathogen or eradication of harmful effects of the pathogen. As used herein “inhibit” refers to a reduction in an amount of a pathogen or a reduction in harmful effects of the pathogen. As used herein “kill” refers to the destruction of a pathogen or the permanent and irreversible elimination of the capacity thereof to proliferate or reproduce. As used herein “slow” refers to reducing the spread of a pathogen or reducing the rate at which harmful effects of the pathogen are established or increase. The terms mitigate, eliminate, inhibit, kill, slow, control, or prevent can include partial or complete mitigation, elimination, inhibition, death, slowing, control, or prevention of the pathogen or of harmful effects of the pathogen. For example, the mitigation, elimination, inhibition, death, slowing, control, or prevention can be of 10%, 20%, 30%, 40%, 50%, 60%, 70%, 80%, 90%, 100%, or an amount within a range defined by any two of the aforementioned values.

As used herein, “obtaining” as in “obtaining an agent” includes synthesizing, purchasing, or otherwise acquiring the agent.

By “pathogen” is meant an organism that causes a disease in a plant. In some embodiments, the pathogen is a fungal pathogen. In embodiments, the fungal pathogen is Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahliae. In some embodiments, the disease is white rot or gray mold. In some embodiments, the fungal pathogen is adversely affecting the growth of plants, the appearance of plants, the production and yield of plant-based food, the appearance of plant-based food, the preservation of plant-based food, the cultivation of plants. In some embodiments, the pathogen is any and all forms of anthracnose or any and all types of Botrytis, Fusarium (including F. oxysporum f. sp. fragariae, Cubense or F. solani), Thielavopsis (root rot), Mycosphaerella (including M. fijiensis and M. musicola), Verticillium (including V. dahlia), Macrophomina phaseolina, Phytophthora cactorum, Magnaporthe grisea, Phythium, Sclerotinia sclerotiorum, Sclerotium cepivorum (alternatively, Stromatinia cepivora), Ustilago, Rhizoctonia (including R. solani), Cladosporium, Colletotrichum (including C. coccodes, C. acutatum, C. truncatum, or C. gloeosporoides), Trichoderma (including T. viride or T. harzianum), Helminthosporium (including H. solani), Alternaria (including A. solani or A. alternata), Aspergillus (including A. niger or A. fumigatus), Phakospora pachyrhizi, Puccinia, oomycetes (including Phytophthora), and Armillaria. In some embodiments, the plant pathogen belongs to the family class Leotiomycetes, to the order Helotiales, and/or to the family Sclerotiniaceae.

By “parts per million (ppm)” is meant a unit of concentration equivalent to mg/L or g/m³, where density of a liquid is estimated at about 1 g/ml, or to mg/kg. For example, 1 L of an aqueous solution containing 100 mg lactate may be described as containing 100 ppm lactate. As a further example, a 1 kg soil sample containing 100 mg lactate may be described as containing 100 ppm lactate.

The term “plant” includes all organisms of the plant kingdom, as well as their cells, tissues, and products. Accordingly, the term plant includes seeds, leaves, stems, roots, fruit, and the like

As used herein, the terms “prevent,” “preventing,” “prevention,” “prophylactic treatment” and the like refer to reducing the probability of developing a disease (e.g., white rot, gray mold) in a plant or soil, that does not have, but is at risk of or susceptible to developing the disease.

By “reduces” is meant a negative alteration of at least 5%, 10%, 25%, 50%, 75%, or 100%.

By “reference” is meant a standard or control condition. In one embodiment, a reference is a plant, soil, or other medium that comprises a fungal pathogen, but that is not contacted with a composition of the invention. In embodiments, the fungal pathogen is Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahliae. In embodiments, the composition of the invention comprises lactate and/or acetate.

By “sterile composition” is meant a composition free from the presence of viable organisms. The term “organism” includes fungal pathogens, non-limiting examples of which include Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum,, or Verticillium dahlia.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50

As used herein, “soil” refers to a composition that functions to provide structural support to plants and functions as a source of water and nutrients for the plants. A soil can contain a mixture of inorganic (e.g., sand, silt, clay, gravel) and organic materials. The soil can contain particles greater than 2 mm in diameter (gravel), particles from about 0.2 mm in diameter to about 2 mm in diameter (coarse sand), particles from about 0.02 mm in diameter to about 0.2 mm in diameter (fine sand), particles from about 0.002 mm in diameter to about 0.02 mm in diameter (silt), particles of less than 0.002 mm in diameter (clay) or various combinations thereof.

As used herein, the terms “treat,” treating,” “treatment,” and the like refer to reducing or ameliorating a disease from a soil or plant.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 presents three plots showing the influence of organic acid compositions on the growth of Sclerotium cepivorum and Botrytis cinerea. In FIG. 1, n.s. indicates “not significant”, ppm indicates “parts per million”; L and (S) indicate levorotatory and sinister rotatory, respectively, and asterisks are used to indicate degrees of statistical significance according to standard conventions. 2-fold concentrated compositions of lactate and acetate were diluted 1:1 with a deionized water + agar solution to prepare agar plates

FIGS. 2A-2E are a plot and images of petri plates inoculated with Sclerotinia sclerotiorum demonstrating that compositions containing lactate and L-lactic acid suppresses growth of the fungal pathogen. The images presented in FIGS. 2A-2D were taken at 2, 3, 4, and 7 days post-inoculation, respectively. In each of FIGS. 2A-2D, the first (top) panel is an image of a negative control petri plate containing water (H2O) instead of the biocontrol agent or a composition containing lactate and L-lactic acid, the second panel is an image of a petri plate containing the biocontrol agent (BCA), and the third (last) panel is an image of a petri plate treated with a composition containing acetate and L-lactic acid. For scale, a centimeter ruler is shown in each image. FIG. 2E provides a plot of fungal colony area over time. Error bars represent one standard deviation from the mean. Throughout the figures “AC” is short for “acetate and L-lactic acid”.

FIGS. 3A-3E are a plot and images of petri plates inoculated with Sclerotinia minor demonstrating that compositions containing lactate and L-lactic acid suppresses growth of the fungal pathogen. The images presented in FIGS. 3A-3D were taken at 2, 3, 4, and 7 days post-inoculation, respectively. In each of FIGS. 3A-3D the first (top) panel is an image of a negative control petri plate containing water (H2O) instead of the biocontrol agent or a composition containing lactate and L-lactic acid, the second panel is an image of a petri plate containing the biocontrol agent (BCA), and the third (last) panel is an image of a petri plate treated with a composition containing acetate and L-lactic acid. FIG. 3E provides a plot of fungal colony area over time. Error bars represent one standard deviation from the mean. Throughout the figures “AC” is short for “acetate and L-lactic acid”.

FIGS. 4A-4D are a plot and images of petri plates inoculated with Pythium uncinulatum demonstrating that compositions containing lactate and L-lactic acid suppresses growth of the fungal pathogen. The images presented in FIGS. 4A-4C were taken at 3, 4, and 7 days post-inoculation, respectively. In each of FIGS. 4A-4C, the first (top) panel is an image of a negative control petri plate containing water (H2O) instead of the biocontrol agent or a composition containing lactate and L-lactic acid, the second panel is an image of a petri plate containing the biocontrol agent (BCA), and the third (last) panel is an image of a petri plate treated with a composition containing acetate and L-lactic acid, each individually at a concentration of 800 ppm.. FIG. 4D provides a plot of fungal colony area over time. Error bars represent one standard deviation from the mean. Throughout the figures “AC” represents “acetate and L-lactic acid”.

FIGS. 5A-5H are bar graphs showing growth of the indicated species on potato dextrose agar with and without addition of the biocontrol agent or of a composition of lactate and L-lactic acid, each individually at a concentration of 800 ppm. Growth was evaluated four and seven days following inoculation of the potato dextrose agar (N=3). 0.2 sq cm was the area of the agar plug used to inoculate the petri plates and, therefore, an area of 0.2 sq cm corresponds to zero growth. 56.7 sq cm was the area of the petri dish. Error bars are equal to one standard deviation.

### DETAILED DESCRIPTION OF THE INVENTION

The invention features compositions and methods that are useful for inhibiting growth and/or survival of a fungal pathogen (e.g., Botrytis cinerea, Colletotrichum acutatum, Fusarium oxysporum f. sp. fragariae, Macrophomina phaseolina, Phytophthora cactorum. Pythium uncinulatum, Rhizoctonia solani, Sclerotinia minor, Sclerotium cepivorum, Sclerotinia sclerotiorum, or Verticillium dahliae).

The present invention is based, at least in part, upon the discovery that compositions containing lactate and acetate are useful for the inhibition of a fungal pathogen (e.g., Botrytis cinerea, Colletotrichum acutatum, Fusarium oxysporum f. sp. fragariae, Macrophomina phaseolina, Phytophthora cactorum, Pythium uncinulatum, Rhizoctonia solani, Sclerotinia minor, Sclerotium cepivorum, Sclerotinia sclerotiorum, or Verticillium dahliae) in soils and/or on the surfaces of plants.

### Compositions

The invention provides compositions used for inhibiting the growth and/or survival of a fungal pathogen (e.g., Botrytis cinerea, Colletotrichum acutatum, Fusarium oxysporum f. sp. fragariae, Macrophomina phaseolina, Phytophthora cactorum. Pythium uncinulatum, Rhizoctonia solani, Sclerotinia minor, Sclerotium cepivorum, Sclerotinia sclerotiorum, or Verticillium dahliae). In embodiments, the compositions comprise lactate and acetate (e.g., L-lactate) and are formulated for agricultural use.

The compositions can comprise lactate and acetate at a particular molar ratio of lactate to acetate (lactate:acetate). The compositions can comprise lactate and acetate at a particular mass ratio of lactate to acetate (lactate:acetate). In some embodiments, the mass ratio or molar ratio of lactate to acetate (lactate:acetate) is from about 1:10 to about 10:1, from about 1:6 to about 6:1, from about 1:4 to about 4:1, from about 1:6 to about 1:1, from about 1:4 to about 1:1, from about 1:3 to about 1:1, from about 6:1 to about 1:1, or from, about 4:1 to about 1:1. In some embodiments, the compositions comprises about or at least about 50 ppm lactate, 75 ppm lactate, 100 ppm lactate, 125 ppm lactate, 150 ppm lactate, 175 ppm lactate, 200 ppm lactate, 300 ppm lactate, 400 ppm lactate, 500 ppm lactate, 600 ppm lactate, 700 ppm lactate, 800 ppm lactate, 900 ppm lactate, 1,000 ppm lactate, 1,100 ppm lactate, 1,200 ppm lactate, 1,300 ppm lactate, 1,400 ppm lactate, 1,500 ppm lactate, 1,600 ppm lactate, 1,700 ppm lactate, 1,800 ppm lactate, 1,900 ppm lactate, 2,000 ppm lactate, 2,200 ppm lactate, 2,300 ppm lactate, 2,400 ppm lactate, 2,500 ppm lactate, 3,000 ppm lactate, 3,500 ppm lactate, 4,000 ppm lactate, 4,500 ppm lactate, 5,000 ppm lactate, or 5,500 ppm lactate. In some embodiments, the compositions comprises not more than about 50 ppm lactate, 75 ppm lactate, 100 ppm lactate, 125 ppm lactate, 150 ppm lactate, 175 ppm lactate, 200 ppm lactate, 300 ppm lactate, 400 ppm lactate, 500 ppm lactate, 600 ppm lactate, 700 ppm lactate, 800 ppm lactate, 900 ppm lactate, 1,000 ppm lactate, 1,100 ppm lactate, 1,200 ppm lactate, 1,300 ppm lactate, 1,400 ppm lactate, 1,500 ppm lactate, 1,600 ppm lactate, 1,700 ppm lactate, 1,800 ppm lactate, 1,900 ppm lactate, 2,000 ppm lactate, 2,200 ppm lactate, 2,300 ppm lactate, 2,400 ppm lactate, 2,500 ppm lactate, 3,000 ppm lactate, 3,500 ppm lactate, 4,000 ppm lactate, 4,500 ppm lactate, 5,000 ppm lactate, or 5,500 ppm lactate. In some embodiments, the composition comprises about or at least about 50 ppm acetate, 75 ppm acetate, 100 ppm acetate, 125 ppm acetate, 150 ppm acetate, 175 ppm acetate, 200 ppm acetate, 300 ppm acetate, 400 ppm acetate, 500 ppm acetate, 600 ppm acetate, 700 ppm acetate, 800 ppm acetate, 900 ppm acetate, 1,000 ppm acetate, 1,100 ppm acetate, 1,200 ppm acetate, 1,300 ppm acetate, 1,400 ppm acetate, 1,500 ppm acetate, 1,600 ppm acetate, 1,700 ppm acetate, 1,800 ppm acetate, 1,900 ppm acetate, 2,000 ppm acetate 2,500 ppm acetate, 3,000 ppm acetate, 3,500 ppm acetate, 4,000 ppm acetate, 4,500 ppm acetate, 5,000 ppm acetate, or 5,500 ppm acetate. In some embodiments, the composition comprises not more than about 50 ppm acetate, 75 ppm acetate, 100 ppm acetate, 125 ppm acetate, 150 ppm acetate, 175 ppm acetate, 200 ppm acetate, 300 ppm acetate, 400 ppm acetate, 500 ppm acetate, 600 ppm acetate, 700 ppm acetate, 800 ppm acetate, 900 ppm acetate, 1,000 ppm acetate, 1,100 ppm acetate, 1,200 ppm acetate, 1,300 ppm acetate, 1,400 ppm acetate, 1,500 ppm acetate, 1,600 ppm acetate, 1,700 ppm acetate, 1,800 ppm acetate, 1,900 ppm acetate, 2,000 ppm acetate 2,500 ppm acetate, 3,000 ppm acetate, 3,500 ppm acetate, 4,000 ppm acetate, 4,500 ppm acetate, 5,000 ppm acetate, or 5,500 ppm acetate.

In some embodiments, a composition of the invention comprises 175 ppm L-lactate and 600 ppm acetate, 600 ppm L-lactate and 600 ppm acetate, 175 ppm L-lactate and 800 ppm acetate, or 800 ppm L-lactate and 800 ppm acetate.

The composition can be substantially free of D-lactate. In some contexts, it can be beneficial to ensure the absence of D-lactate or only low concentrations of D-lactate in the compositions of the invention to prevent D-lactate from functioning as a carbon source for a pathogenic fungus. In some embodiments, a composition “substantially free” of D-lactate comprises less than about 1% (wt/wt), 2% (wt/wt), 3% (wt/wt), 4% (wt/wt), 5% (wt/wt), 6% (wt/wt), 7% (wt/wt), 8% (wt/wt), 9% (wt/wt), 10% (wt/wt), 11% (wt/wt), 12% (wt/wt), 13% (wt/wt), 14% (wt/wt), 15% (wt/wt), 16% (wt/wt), 17% (wt/wt), 18% (wt/wt), 19% (wt/wt), or 20% (wt/wt) D-lactate.

The compositions may be prepared by mixing lactate and acetate with agriculturally acceptable carriers and/or additives. Non-limiting examples of carriers and/or additives include extenders, solvents, diluents, dyes, wetters, dispersants, emulsifiers, antifoaming agents, nutrients, preservatives, secondary thickeners, adhesives, and/or water. Formulations of the present invention may include agriculturally acceptable carriers, which are inert formulation ingredients added to formulations to improve recovery, efficacy, or physical properties and/or to aid in packaging and administration. Carriers may include anti-caking agents, anti-oxidation agents, bulking agents, and/or protectants. Examples of useful carriers include polysaccharides (starches, maltodextrins, methylcelluloses, proteins, such as whey protein, peptides, gums), sugars (lactose, trehalose, sucrose), lipids (lecithin, vegetable oils, mineral oils), salts (sodium chloride, calcium carbonate, sodium citrate), silicates (clays, amorphous silica, fumed/precipitated silicas, silicate salts), waxes, oils, alcohol and surfactants.

Further non-limiting examples of carriers include a natural or synthetic, organic or inorganic substance which is mixed or combined with lactate and acetate for better applicability, in particular for application to plants or plant parts, soils, or seeds. The support or carrier, which may be solid or liquid, is generally inert and should be suitable for use in agriculture. Suitable solid or liquid carriers/supports include for example ammonium salts and natural ground minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral oils and vegetable oils, and also derivatives and various combinations thereof. It is also possible to use mixtures of such supports or carriers. Solid supports/carriers suitable for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks. Suitable liquefied gaseous extenders or carriers are liquids which are gaseous at ambient temperature and under atmospheric pressure, for example aerosol propellants, such as butane, propane, nitrogen and carbon dioxide. Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils and waxes, optionally modified. If the extender used is water, it is also possible for example, to use organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

In some embodiments, the composition may include components that facilitate the application of the composition to a plant or soil. The application of a composition of the invention to soil may be performed by drenching, incorporation into soil, or by droplet application. The compositions may also be applied directly to plant roots or seeds (e.g., via immersion, dusting, or spraying). To assist in the application, the compositions can be in the form of liquid solutions, emulsions, wettable powders, suspensions, powders, dusts, pastes, soluble powders, granules, or suspension-emulsion concentrates.

In some embodiments, the composition may be a sterile liquid solution. In some embodiments, the composition may contain a liquid diluent or solvent (e.g., water). A non-limiting example of a diluent is an aqueous solution that is compatible with plant, soil, aquaculture, or livestock application, such that the composition does not adversely affect the growth of plants, aquatic life, or livestock. The carrier may be a liquid. The carrier may improve the stability, handling, storage, shipment, or application properties of the composition.

In some embodiments, the compositions further include a surfactant In some embodiments, the surfactant includes glycerol, alkylbenzenesulfonate, ammonium lauryl sulfate, sodium lauryl sulfate (SLS), sodium dodecyl sulfate (SDS), sodium laureth sulfate, sodium lauryl ether sulfate (SLES), sodium myreth sulfate, dioctyl sodium sulfosuccinate, perfluorooctane sulfonate, perfluorobutanesulfonate, alkyl- aryl ether phosphates, alkyl ether phosphates, sodium stearate, sodium lauroyl sarcosinate, perfluorononanoate, and perfluorooctanoate. In some embodiments, the compositions include an emulsifier present in an amount of ranging from about 0.001% to about 10%, such as 0.001, 0.005, 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, or 10%, or in an amount within a range defined by any two of the aforementioned values.

In some embodiments, the surfactant comprises an emulsifier, a dispersing agent or a wetting agent of ionic or non-ionic type or a mixture of such surfactants. Further non-limiting examples of surfactants include polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions.

Additional components may also be included in the compositions, as non-limiting examples, protective colloids, adhesives, nutrients, thickeners, thixotropic agents, penetration agents, stabilizers, sequestering agents.

In some embodiments, the compositions comprise colorants, such as inorganic pigments (e.g, iron oxide, titanium oxide, and Prussian blue), and organic dyes (e.g., alizarin dyes, and azo dyes) and metal phthalocyanine dyes.

In some embodiments, the composition is formulated as a sterile liquid media, a solution, a spray, a mist, a seed coating, an electrostatically charged seed powder, a powder, a powder-like substance, or a freeze-dried powder.

In some embodiments, additional components may be included in compositions, as non-limiting examples, such as benzoids, pyrazines, alcohols, ketones, volatile fatty acids, volatile organic compounds, sulfides and/ or alkenes.In some embodiments, additional nutrient and biostimulant components may be included in compositions, as non-limiting examples, such as nitrogen, potassium, phosphate, as well as beneficial bacterial species and beneficial fungal species.

In some embodiments, the composition may be formulated as a seed coating. In some embodiments, the composition may be a conglomerate mixture with additional nutrients used to coat a plant seed. In some embodiments, the composition protects the plant seed from harmful pathogens, such as fungi, during storage. In some embodiments, the composition increases germination rates, increases seedling survival, and/or increases crop yields.

In some embodiments, the composition may be formulated for application to a crop, a plant, a tree, turf, or soil by spraying, misting, soaking, watering, soil drenching, crop-dusting, or otherwise applying the composition to the soil, plants, the portion of the plants, or components of the plants. In some embodiments, the composition is applied to the plant itself, such as to the leaves, stem, trunk, stalk, flowers, branches, fruits, roots, shoots, buds, rhizome, seeds, or other portions of the plant, or it is applied to the soil in which or around which the plant is being cultivated. In some embodiments, the composition is formulated as a solution that is applied to the plant or to plant parts, such as applied to harvested seeds, leaves, stem, trunk, stalk, flowers, branches, fruits, roots, shoots, buds, rhizome, or other portions of the plant, or to the soil in which or around which the plant is being cultivated. In some embodiments, the composition is applied to turf grass. In some embodiments, the composition is freeze-dried or otherwise reduced to a solid or powder through an evaporative process. In some embodiments, the composition is formulated together with a fertilizer or micro-nutrient for application to a plant or soil. Such fertilizers or nutrients may include, for example, trace minerals, phosphorus, potassium, sulfur, manganese, magnesium, calcium, and/or any one or more of a trace element. In some embodiments, the composition is formulated as a concentrated composition that may be diluted prior to application. For example, the composition may be formulated as a liquid concentrate that may be diluted with a solution, such as with water, or it may be formulated as a solid, such as a powder, for dissolution in a solution, such as water. In some embodiments, the composition may be formulated as a ready-to-use composition. For example, the composition may be formulated as a solution that includes the appropriate concentrations of component parts for direct application to a plant or may be formulated as a solid for direct application to a plant.

In any of the embodiments of the compositions provided herein, formulations may be developed as adjuvants to be applied concurrently with existing commercial products to enable and/or enhance their effectiveness.

In any of the embodiments of the compositions provided herein, the compositions may be non-toxic and include component parts that exhibit no toxic effects to humans, to the soil or plant that is being treated, or to the environment, including no toxicity to groundwater, flora, or fauna. Components suitable for use in any of the embodiments of the compositions provided herein can result in improved agricultural health, including improved plant health and/or improved crop production, or improved aquaculture or livestock health. Furthermore, embodiments of the compositions provided herein enable ease in application of the compositions.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, soluble concentrate, soluble powder, liquid solution, suspension concentrate (flowable concentrate), water dispersible granules or tablets, water dispersible powder for slurry treatment, water soluble granules or tablets, water soluble powder, and wettable powder.

These compositions include not only compositions which are ready to be applied to a plant, seed, or soil to be treated by means of a suitable device, such as a spraying or dusting device, but also concentrated commercial compositions (i.e., concentrates) which must be diluted before they are applied to a soil or plant.

In some embodiments, the composition is a soil or a potting soil. The soil or potting soil may be disposed in, to provide non-limiting examples, a planter, a pot, a bag, or a sealed bag.

### Methods of Delivery

In some embodiments, the methods include treating soil, crop plant, tree, turf, or an ornamental plant having a fungal disease (e.g., white rot, gray mold) with the compositions described herein.

In some embodiments, the composition is applied to the soil, crop plant, tree, turf, or ornamental plant until a target concentration of lactate is attained in the soil and/or on a surface of the plant. In some embodiments, the target concentration of lactate in the soil and/or on the surface of the plant is about or at least about 50 ppm lactate, 75 ppm lactate, 100 ppm lactate, 125 ppm lactate, 150 ppm lactate, 175 ppm lactate, 200 ppm lactate, 300 ppm lactate, 400 ppm lactate, 500 ppm lactate, 600 ppm lactate, 700 ppm lactate, 800 ppm lactate, 900 ppm lactate, 1,000 ppm lactate, 1,100 ppm lactate, 1,200 ppm lactate, 1,300 ppm lactate, 1,400 ppm lactate, 1,500 ppm lactate, 1,600 ppm lactate, 1,700 ppm lactate, 1,800 ppm lactate, 1,900 ppm lactate, 2,000 ppm lactate, 2,500 ppm lactate, 3,000 ppm lactate, 3,500 ppm lactate, 5,000 ppm lactate, or 5,500 ppm lactate. In some embodiments, the target concentration of lactate in the soil and/or on the surface of the plant is not greater than about 50 ppm lactate, 75 ppm lactate, 100 ppm lactate, 125 ppm lactate, 150 ppm lactate, 175 ppm lactate, 200 ppm lactate, 300 ppm lactate, 400 ppm lactate, 500 ppm lactate, 600 ppm lactate, 700 ppm lactate, 800 ppm lactate, 900 ppm lactate, 1,000 ppm lactate, 1,100 ppm lactate, 1,200 ppm lactate, 1,300 ppm lactate, 1,400 ppm lactate, 1,500 ppm lactate, 1,600 ppm lactate, 1,700 ppm lactate, 1,800 ppm lactate, 1,900 ppm lactate, 2,000 ppm lactate, 2,500 ppm lactate, 3,000 ppm lactate, 3,500 ppm lactate, 5,000 ppm lactate, or 5,500 ppm lactate. In some embodiments, the composition is applied to the soil and/or the surface of the plant until a target concentration of acetate is attained in the soil and/or on the surface of the plant. In some embodiments, the target concentration of acetate in the soil and/or on the surface of the plant is about or at least about 50 ppm acetate, 75 ppm acetate, 100 ppm acetate, 125 ppm acetate, 150 ppm acetate, 175 ppm acetate, 200 ppm acetate, 300 ppm acetate, 400 ppm acetate, 500 ppm acetate, 600 ppm acetate, 700 ppm acetate, 800 ppm acetate, 900 ppm acetate, 1,000 ppm acetate, 1,100 ppm acetate, 1,200 ppm acetate, 1,300 ppm acetate, 1,400 ppm acetate, 1,500 ppm acetate, 1,600 ppm acetate, 1,700 ppm acetate, 1,800 ppm acetate, 1,900 ppm acetate, 2,000 ppm acetate, 2,500 ppm acetate, 3,000 ppm acetate, 3,500 ppm acetate, 5,000 ppm acetate, or 5,500 ppm acetate. In some embodiments, the target concentration of acetate in the soil and/or on the surface of the plant is not more than about 50 ppm acetate, 75 ppm acetate, 100 ppm acetate, 125 ppm acetate, 150 ppm acetate, 175 ppm acetate, 200 ppm acetate, 300 ppm acetate, 400 ppm acetate, 500 ppm acetate, 600 ppm acetate, 700 ppm acetate, 800 ppm acetate, 900 ppm acetate, 1,000 ppm acetate, 1,100 ppm acetate, 1,200 ppm acetate, 1,300 ppm acetate, 1,400 ppm acetate, 1,500 ppm acetate, 1,600 ppm acetate, 1,700 ppm acetate, 1,800 ppm acetate, 1,900 ppm acetate, 2,000 ppm acetate, 2,500 ppm acetate, 3,000 ppm acetate, 3,500 ppm acetate, 5,000 ppm acetate, or 5,500 ppm acetate.

In some embodiments, the target concentration in soil and/or on the surface of the plant is about 87.5 ppm L-lactate and 100 ppm acetate, 175 ppm L-lactate and 200 ppm acetate, 175 ppm L-lactate and 600 ppm acetate, 175 ppm L-lactate and 800 ppm acetate, , 600 ppm L-lactate and 600 ppm acetate, or 800 ppm L-lactate and 800 ppm acetate.

The precise amount of lactate and acetate to be applied to a particular plant or soil in accordance with the invention will depend upon the sensitivities of the particular plant, the method of application, and field conditions such as the quality of the soil. All of these factors can be taken into consideration by one skilled in the art to determine an optimal amount of lactate and acetate to apply to a plant or soil for a particular application. The compositions are applied to a plant or soil in an amount effective to control (e.g., inhibit growth or survival) a pathogen.

Crop plants (e.g., Allium plants such as garlic or onions), trees, turf, and ornamental plants pass through different stages in their growth. For example, onions (an Allium plant) pass through at least three stages during their growth: vegetative, bulbing, and blooming (bolting). In some embodiments, the roots of an Allium plant are contacted with a composition of the invention during the vegetative, bulbing, or blooming stage of plant growth. In some embodiments, the compositions are applied to a plant or soil at the time of planting or prior to the time of planting. The compositions can also be applied once plants are established within the soil. The compositions can be applied to seeds, reproductive vegetative material, seedlings, and/or established plants regardless of their stage of growth.

In some embodiments, the crop plant, tree, turf, soil or ornamental plant is treated for a potential or actual fungal pathogenic disease (e.g., white rot, gray mold). The plant or soil can be outside or inside (e.g., in a greenhouse or other enclosure) The plant could be an ornamental, a crop, or an aquaculture plant. The soil can be soil used for the production of any agricultural or horticultural product, such as cereals, vegetables, fruits, nuts, beans, seeds, herbs, spices, fungi, ornamental plants (e.g., flowers, bushes, turf, and trees), industrial plants, and/or plants grown for feed. In some embodiments, the plant or soil exhibits industrial, commercial, recreational, or aesthetic value. In some embodiments, the compositions of the present invention are used to treat a plant. In some embodiments, the plant is a poinsettia, flowers, lupin, grass, alfalfa, trees, or ivy In some embodiments the plant is a food producing plant. In some embodiments, the plant is a banana, cacao, canola, coffee, bean, cotton, garlic, onion, leek, chive, maize, wheat, rice, corn, leafy greens, potato, tomato, pepper, squash, gourds, cucumber, berry, grape vine or grapes, pome, drupe, citrus, melon, tropical fruit, cotton, nut, soybean, sorghum, cane, cucurbits, onion, aubergine, parsnip, Cannabis (e.g., hemp), herb, tobacco, or pulse plant. The plant can be an Allium plant. Non-limiting examples of Allium plants include Allium sativum, Allium cepa, Allium chinense, Allium stipitatum, Allium schoenoprasum, Allium tuberosum, Allium fistulosum, and Allium ampeloprasum.

In some embodiments, the methods include applying the composition to a plant or to the soil in which the plant is growing. Applying the composition may be achieved by various means, including, for example, by sprinklering, spraying, drenching, soaking, watering, crop-dusting, misting, high-pressure liquid injection, or otherwise applying the composition to the plants or surrounding soil. The composition can be applied using an irrigation system. In some embodiments, the composition is applied to the plant itself, such as to the leaves, stem, trunk, stalk, flowers, branches, fruits, roots, shoots, buds, rhizome, seeds, or other portions of the plant, or it is applied to the soil in which or around which the plant is being cultivated. In some embodiments, the composition is formulated as a seed coating, and the method includes coating a seed with the composition. In some embodiments, the seed coating is an electrostatic seed coating. In some embodiments, the seed coating includes micronutrients. In some embodiments, the seed coating protects the plant seed from harmful pathogens, such as fungi. In some embodiments, the seed coating allows for uniform size of plant seeds for bulk planting techniques. In some embodiments, the seed coating increases germination rates, increases seedling survival, and/or increases crop yields. In some embodiments, the composition is formulated as a powder, and the method includes applying the powder to the plant or to plant parts, such as applied to seeds, leaves, stem, trunk, stalk, flowers, branches, fruits, roots, shoots, buds, rhizome, or other portions of the plant, or to the soil in which or around which the plant is being cultivated. In some embodiments, the composition is formulated together with a fertilizer or nutrient, and the method includes incorporating the composition into the soil through disking or tilling or applying the fertilizer or nutrient to the plant. The compositions of the invention can be applied to a plant seed, to soil within which a plant is growing, to soil in which a plant or seed is about to be planted, to a plant (e.g., plant roots), or to combinations thereof.

Particular concentrations and/or concentration ratios of lactate and acetate may be important to maintaining an optimal degree of biocontrol in soil. Therefore, in some embodiments, the methods of the invention involve monitoring or measuring the concentration of lactate and acetate in a soil and adding a composition of the invention to establish, restore, or maintain target concentrations in the soil. In some embodiments, the concentration of acetate and lactate in the soil is monitored continuously, hourly, daily, weekly, monthly, bi-monthly, or every four months. In some embodiments, if the concentration of acetate and/or lactate falls below a target concentration in the soil, acetate and/or lactate is added to the soil to bring the concentration back to a target concentration and/or to within a target concentration range. Representative methods for measuring lactate and acetate concentrations are discussed herein below.

In embodiments the composition is applied to a soil and/or plant multiple times. In embodiments, the soil and/or plant is contacted with the composition about or at least about 1, 2, 3, 4, 5, 6, 7, 8, 9, 10, 11, 12, 13, 14, 15, 16, 17, 18, 19, or 20 times. In various embodiments, each contacting is spaced from the previous contacting by a time interval individually ranging from about or at least about 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days 24 days, or 25 days. In various embodiments, the composition is applied to the soil before the time of planting by a time interval ranging from about or at least about 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days 24 days, or 25 days before planting. In embodiments, the composition is applied to the soil and/or plant at time of planting. In embodiments, the composition is applied to the soil and/or plant at 10 days, 14 days, 28 days, and 42 days after planting. In embodiments, the composition is applied by spray or drip application. In embodiments, the composition is applied at 14 days, 30 days, 36 days, and 42 days post-planting. In embodiments, a last application of the composition is by drip application.

In embodiments, application of the composition does not adversely affect the vigor of a plant. In embodiments, the application of the composition is not toxic to a plant.

In some embodiments, the compositions are applied to a plant or soil at a time of planting or prior to the time of planting. The compositions can also be applied once plants are established within the soil. The compositions can be applied to seeds, reproductive vegetative material, seedlings, and/or established plants.

### Soil Characterization

The concentration of lactate and acetate in a liquid or soil sample can be determined by a variety of methods familiar to one of ordinary skill in the art including, to provide non-limiting examples, high performance liquid chromatography (HPLC) (e.g., Lawongsa, et al., “Determination of organic acids in soil by high performance liquid chromatography,” Soil Sci. Plant. Nutr. 33:299-302 (1987)), ion chromatography (e.g., Baziramakenga, et al., “Determination of organic acids in soil extracts by ion chromatography,” Soil Biology and Biochemistry, 27:349-356 (1995)), and mass spectroscopy (e.g., pyrolysis-field ionization mass spectroscopy, as described in Adeleke, “Origins, roles and fate of organic acids in soils: a review,” South African Journal of Botany, 108:393-406 (2017)).

In one embodiment of a method for quantitation of organic acids in a soil sample, the acids can be extracted from the sample using an acidic extractant, such as KH₂PO₄ or NaH₂PO₄. The extract can be analyzed using high performance liquid chromatography (HPLC) or gas chromatography (GC).

### Pathogen Characterization

In some embodiments, the methods of the disclosure include detecting the presence of a pathogenic fungus in soil or on a plant. The method can further include adding a composition of the present invention to the soil or contacting the plant with the composition only if presence of the pathogenic fungus is detected. One of skill in the art will be able to determine a suitable method for determining the presence of a fungal pathogen in soil or on a plant. Non-limiting examples of methods for detecting the presence of a fungal pathogen in soil or on a plant include visual inspection, microscopic techniques, next generation sequencing, DNA microarrays, macroarrays (e.g., membrane-based DNA macroarrays, as described by Lievens, et al., “Fungal plant pathogen detection in plant and soil samples using DNA macroarrays,” Methods Mol. Biol. 835:491-507 (2012), which is incorporated herein by reference in its entirety for all purposes), and PCR. The methods of the present invention can include monitoring effectiveness of a compositions of the present invention in inhibiting, controlling, reducing, or eliminating growth of a plant pathogenic fungus by measuring a titer of the pathogenic fungus in soil or on a plant before, during, and/or after application of the composition to the soil or plant. In some embodiments, a method of the disclosure includes modifying the concentration of lactate or acetate in a composition applied to a soil or plant to optimize a reduction in titer or growth rate of a pathogenic fungus in the soil or in or on the plant.

In one embodiment, the method of the disclosure includes determining the composition of a microbial community associated with a plant or soil treated by the method. In some embodiments, the composition of the microbial community is determined using techniques familiar to one of skill in the art including, as non-limiting examples, PCR, next generation sequencing, and DNA microarrays. In some embodiments, the composition of the microbial community is determined by sequencing a 16S and/or 18S rRNA gene.

### Kits

This disclosure provides a kit that includes a composition comprising lactate (e.g., L-lactate) and acetate. In some embodiments, the kit comprises an applicator. In some embodiments, the kit is a ready-to-use kit, wherein the composition included in the kit is ready to use by the user without further alterations. In some embodiments, the composition is provided in the kit in a container for application to a plant or soil. In some embodiments, the container is a spray applicator containing the composition. In some embodiments, the composition is a concentrated liquid, or a solid. In such embodiments, the composition may be added to a liquid, such as water, to dilute the concentrated liquid or to dissolve the solid composition. In some embodiments, the composition is a diluted composition. In some embodiments, the spray applicator is configured for industrial, commercial, home-gardener, or recreational purposes. In some embodiments, the kit includes a dispensing apparatus, such as a nozzle, a valve, a sprayer, or any other apparatus capable of dispensing the compositions described herein.

If desired, the kit further contains instructions for using the compositions and/or administering the compositions. In particular embodiments, the instructions include at least one of the following: description of the components of the composition; application amounts and techniques; precautions; warnings; counter-indications; instructions on how to monitor soil organic acid compositions; instructions on how to monitor soil for the presence of a pathogenic fungus; instructions on how to determine composition of a soil microbiome; and/or references. The instructions may be printed directly on components of the kit or provided as a separate sheet, pamphlet, card, or folder supplied with the kit. The instructions can be provided in digital form on a portable data storage medium (e.g., a compact disk or USB drive) or stored remotely on a server that can be accessed remotely.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Effects of Organic Acid Treatments on the In Vitro Growth of Two Plant Fungal Pathogens

Sclerotium cepivorum, the causative agent for white rot (also known as Allium root rot), and Botrytis cinerea, the causative agent for gray mold, are two agriculturally important pathogenic fungi. S. cepivorum is a soil-borne plant pathogen that affects plants in the Allium genus (e.g., onions, garlic, and leeks). B. cinerea is a plant pathogen that affects a variety of plant species including grapes, tomatoes, rhubarb, cannabis, and strawberries. B. cinerea can attack crops pre- and post-harvest and is considered one of the most important post-harvest pathogens in fresh fruits and vegetables. Existing treatments for these fungi use millions of pounds of synthetic chemicals with unsustainable environmental and human health consequences. Thus, experiments were completed to evaluate the efficacy of compositions of environmentally sustainable and nontoxic organic acids in controlling growth of these fungal pathogens.

Lactate and acetate were identified as metabolites important to healthy soils. Therefore, experiments were completed to evaluate the efficacy of lactate and acetate to inhibit Sclerotium cepivorum and Botrytis cinera.

In a first round of experiments, the effect of acetate, and dextrorotary (d), levorotary (1), and racemic mixtures of enantiomers of lactate alone, or in combination with acetate was evaluated on the growth of Sclerotium cepivorum and Botrytis cinera (Tables 1A and 1B). The absolute configuration of the stereocenters of lactate are designated as R or S. Acetate significantly inhibited growth and the inhibition increased with acetate concentration at the concentrations evaluated in Tables 1A and 1B (below). For S. cepivorum, lactate L (S) and lactate DL (racemic mixture) inhibited growth, whereas lactate D (R) appeared to act as a carbon source. Lactate DL and acetate inhibited growth of both S. cepivorum and B. cinerea. Based upon these results, further experiments were conducted to evaluate higher concentration mixtures of Lactate L (S) and Acetate (Tables 2A and 2B).

The effects of higher concentrations of acetate and lactate L (S) on growth was evaluated (Tables 2A and 2B). The inhibitory effect of acetate on S. cepivorum growth increased with acetate concentration at all concentrations evaluated (Tables 1A, 1B, 2A, and 2B). While Lactate L (S) alone was not effective at the concentrations evaluated in suppressing growth of S. cepivorum. Lactate L (S) and acetate were highly effective in inhibiting growth with complete suppression of growth at lactate L (800 ppm) and acetate (800 ppm). Therefore, a synergistic growth inhibitory effect was observed between lactate L (S) and acetate. Thus, a final round of experiments was completed to further investigate the effect of various lactate L (S) + acetate compositions on growth of S. cepivorum.

All levo-lactate (L-lactate) mixtures with acetate showed statistically significant efficacy against S. cepivorum at levels of ≥50% (Table 3). Increasing acetate concentrations, when mixed with 175 ppm L-lactate, from 600 ppm to 800 ppm increased efficacy. Nearly complete growth inhibition was attained when L-lactate levels of 600 ppm or 800 ppm were mixed with 600 ppm acetate or with 800 ppm acetate, respectively. High concentrations of both organic acids resulted in maximal growth inhibition.

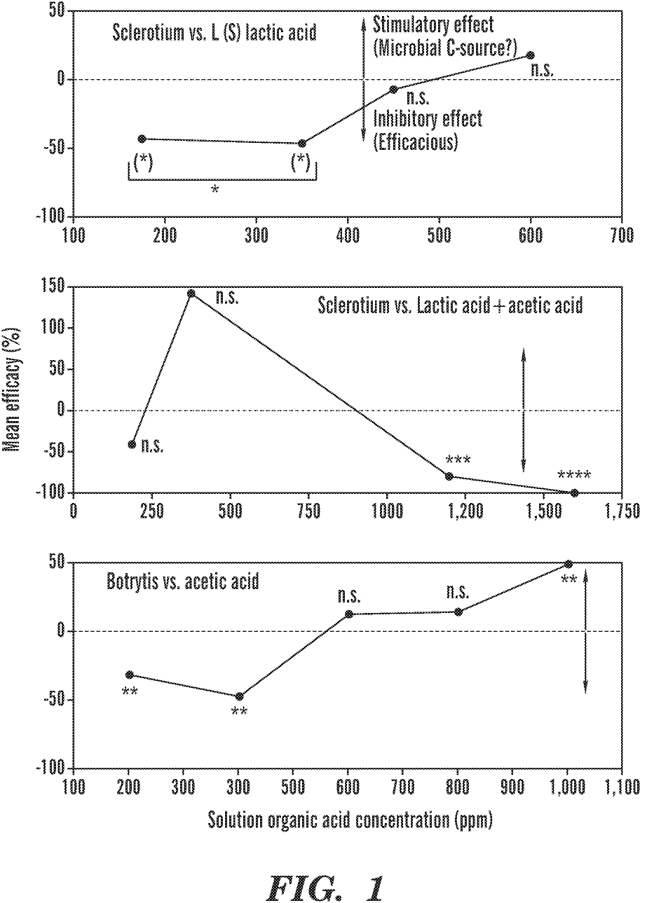

A summary of the inhibitory effect of L (S) lactic acid, lactic acid and acetic acid, and acetic acid on growth of B. cinera and S. cepivorum is provided in FIG. 1. L (S) lactic acid inhibited growth of S. cepivorum at lower concentrations, but served as a carbon source at higher concentrations. Analogously, acetic acid inhibited growth of B. cinera at lower concentrations, but served as a carbon source at higher concentrations. Levo-lactate and acetate in combination acted synergistically to inhibit growth of S. cepivorum.

TABLE 1A

Effects of organic acid treatments on the in vitro growth of the plant fungal pathogen Sclerotium cepivorum (white rot) -- viewed 7 days after inoculation of plates (mean±SE (standard error), n=4 plates). Acid efficacy expressed as a percentage of the mean growth in control plates if growth in treated plates was significantly different (P<0.05 vs. n.s. (not significant) for P≥0.05) from growth in control plates

| Organic acid (OA) species (sp) | Sclerotium Control | Treated | Treated ppm | % Efficacy | P | P-OA sp |
|---|---|---|---|---|---|---|
| Acetate | 2.95 ± 0.97 | 3.63 ± 2.30 | 200 | n.s. [23] | 0.7938 | 0.7622 |
| | 4.32 ± 1.60 | 2.62 ± 1.85 | 400 | n.s. [-39] | 0.5128 | |
| Lactate DL (RS racemic mix) | 7.38 ± 0.26 | 2.06 ± 1.18 | 175 | -72 | 0.0400 * | 0.7379 |
| | 3.17 ± 0.03 | 6.71 ± 2.90 | 350 | n.s. | 0.4606 | |
| Lactate D (R) | 2.50 ± 0.65 | 14.09 ± 5.75 | 175 | n.s. | 0.2506 | 0.1488 |
| | 7.48 ± 3.73 | 10.68 ± 3.19 | 350 | n.s. | 0.5792 | |
| Lactate L (S) | 10.09 ± 0.14 | 5.73 ± 1.72 | 175 | [-43] | 0.1664 | 0.0216 * |
| | 9.65 ± 0.43 | 5.17 ± 1.85 | 350 | [-46] | 0.1599 | |
| Lactate DL (175 ppm) + Acetate (200 ppm) | 2.18 ± 0.30 | 5.28 ± 2.23 | 375 | n.s. [142] | 0.4060 | 0.8793 |
| Lactate DL (87.5 ppm) + Acetate (100 ppm) | 6.06 ± 3.67 | 3.59 ± 0.47 | 187.5 | n.s. [-41] | 0.3531 | |

TABLE 1B

Effects of organic acid treatments on the in vitro growth of the plant fungal pathogen Botrytis cinerea (gray mold) -- viewed 7 days after inoculation of plates (mean±SE (standard error), n=4 plates). Acid efficacy expressed as a percentage of the mean growth in control plates if growth in treated plates was significantly different (P<0.05 vs. n.s. (not significant) for P≥0.05) from growth in control plates

| Organic acid (OA) species (sp) | Botrytis Control | Treated | Treated ppm | % Efficacy | P | P-OA sp |
|---|---|---|---|---|---|---|
| Acetate | 24.03 ± 1.71 | 16.19 ± 1.09 | 200 | -33 | 0.0083 ** | 0.0000 *** |
| | 24.30 ± 2.18 | 12.37 ± 0.79 | 400 | -49 | 0.0021 ** | |
| Lactate DL (RS racemic mix) | 17.34 ± 1.54 | 16.81 ± 1.23 | 175 | n.s. | 0.8124 | 0.4307 |
| | 19.65 ± 2.65 | 26.15 ± 3.37 | 350 | n.s. | 0.2884 | |
| Lactate D (R) | 15.71 ± 2.17 | 23.42 ± 1.23 | 175 | 49 | 0.0276 * | 0.0127 * |
| | 17.64 ± 3.5 | 23.82 ± 2.65 | 350 | [35] | 0.2422 | |
| Lactate L (S) | 17.14 ± 0.25 | 23.59 ± 2.66 | 175 | [38] | 0.1808 | 0.0488 * |
| | 16.25 ± 2.16 | 27.71 ± 4.97 | 350 | [71] | 0.2047 | |
| Lactate DL (175 ppm) + Acetate (200 ppm) | 24.78 ± 0.24 | 15.70 ± 2.32 | 375 | -37 | 0.0594 * | 0.0425 * |
| Lactate DL (87.5 ppm) + Acetate (100 ppm) | 21.41 ± 2.71 | 18.39 ± 2.51 | 187.5 | [-14] | 0.5040 | |

TABLE 2A

Effects of organic acid treatments on the in vitro growth of the plant fungal pathogen Sclerotium cepivorum (white rot) -- viewed 7 days after inoculation of plates (mean±SE (standard error), n=4 plates). Acid efficacy expressed as a percentage of the mean growth in control plates if growth in treated plates was significantly different (P<0.05 vs. n.s. (not significant) for P≥0.05) from growth in control plates

| Organic acid (OA) species (sp) | Sclerotium Control | Treated | Treated ppm | % Efficacy | P | P-OA sp |
|---|---|---|---|---|---|---|
| Acetate | 17.32 ± 2.00 | 2.41 ± 0.57 | 600 | -86 | 0.0001 *** | 0.0001 *** |
| | 17.32 ± 2.00 | 0.51 ± 0.20 | 800 | -97 | 0.0000 *** | |
| Lactate L (S) | 17.32 ± 2.00 | 16.08 ± 3.55 | 450 | n.s.[-7] | 0.8110 | 0.7916 |
| | 17.32 ± 2.00 | 20.50 ± 4.10 | 600 | n.s.[18] | 0.5937 | |
| Lactate L (600 ppm) + Acetate (600 ppm) | 17.32 ± 2.00 | 3.48 ± 0.97 | 1200 | -80 | 0.0004 ** | 0.0001 *** |
| Lactate L (800 ppm) + Acetate (800 ppm) | 17.32 ± 2.00 | 0.00 ± | 1600 | -100 | 0.0000 *** | |

TABLE 2B

Effects of organic acid treatments on the in vitro growth of the plant fungal pathogen Botrytis cinerea (gray mold) -- viewed 7 days after inoculation of plates (mean±SE (standard error), n=4 plates). Acid efficacy expressed as a percentage of the mean growth in control plates if growth in treated plates was significantly different (P<0.05 vs. n.s. (not significant) for P≥0.05) from growth in control plates

| Organic acid (OA) species (sp) | Botrytis Control | Treated | Treated ppm | % Efficacy | P | P-OA sp |
|---|---|---|---|---|---|---|
| Acetate | 20.09 ± 5.19 | 22.25 ± 4.12 | 600 | n.s.[11] | 0.7565 | 0.2094 |
| | 20.09 ± 5.19 | 22.73 ± 4.69 | 800 | n.s.[13] | 0.7298 | |
| | 20.09 ± 5.19 | 29.75 ± 3.76 | 1000 | 48 | 0.0021 ** | |

TABLE 3

Effects of organic acid treatments on the in vitro growth of the plant fungal pathogen Sclerotium cepivorum (white rot) -- viewed 7 days after inoculation of plates (mean±SE, n=4 plates). Acid efficacy expressed as a percentage of the mean growth in control plates if growth in treated plates was significantly different (P<0.05 vs. n.s. for P≥0.05) from growth in control plates

| Organic acid (OA) species (sp) | Sclerotium Control | Treated | Treated ppm | % Efficacy | P |
|---|---|---|---|---|---|
| Lactate L (175 ppm) + Acetate (600 ppm) | 23.28 ± 0.74 | 11.60 ± 1.41 | 775 | -50 | 0.0004 *** |
| Lactate L (175 ppm) + Acetate (800 ppm) | 19.48 ± 2.08 | 6.54 ± 1.24 | 975 | -66 | 0.0003 *** |
| Lactate L (600 ppm) + Acetate (600 ppm) | 21.08 ± 3.16 | 1.87 ± 0.59 | 1200 | -91 | 0.0001 **** |
| Lactate L (800 ppm) + Acetate (800 ppm) | 19.86 ± 1.24 | 1.75 ± 1.56 | 1600 | -91 | 0.0000 **** |

### Example 2: In Vitro Testing of Compositions Containing Acetate and L-Lactic Acid Against Three Plant Pathogens

Various species of Pythium and Sclerotinia fungi are important plant pathogens in agricultural and horticultural industries worldwide. Both fungal groups affect dozens of commercial crops and can cause significant losses of commodity quality, yields, and profit. Pythium species are most often associated with young seedling root rots and plant decline and death. Pythium uncinulatum, causes root rot and plant death of lettuce and has become an economically damaging pathogen in California. Of the various Sclerotinia species, Sclerotinia sclerotiorum and Sclerotinia minor are the two most economically important plant pathogens. Both species have very broad host ranges and cause crown rots of many plants. In addition, Sclerotinia sclerotiorum has an aerial spore stage that results in foliar blights and rots.

A biocontrol agent (produced according to the method described previously in 62/992,364, filed 20 Mar. 2020, the disclosure of which is incorporated herein in its entirety for all purposes) and a composition containing acetate and L-lactic acid were tested for their ability to prevent or inhibit growth of the three plant pathogens.

The biocontrol agent and the composition containing acetate and L-lactic acid were each used to prepare potato dextrose agar (PDA) agarose plates, which also contained streptomycin. The biocontrol agent and the composition containing acetate and L-lactic acid were each individually added at a 1:1 ratio (composition) to PDA (example: 180 ml of streptomycin-PDA mixed with 180 ml of BCA) to prepare two sets of petri plates. As a negative control, a set of Petri plates was also prepared without addition of the biocontrol agent or the composition containing acetate and L-lactic acid (i.e., water was added in place of the biocontrol agent or composition). The prepared petri plates were cooled and then inoculated on the same day that they were prepared. The final concentration of each of acetate and L-lactic acid in the PDA plates were 800 ppm, respectively.

To provide inocula for the experiment, mycelial cultures of each of the fungal pathogens grown on potato dextrose agar were prepared. Fresh PDA plates, prepared as described above, were inoculated with a single 5-mm diameter agar plug taken from the prepared mycelial cultures. Each plug was placed in the middle of the PDA plate being inoculated. Growth of each pathogen was evaluated in triplicate for each of the three treatments (i.e., biocontrol agent, acetate and L-lactic acid composition, and the negative control). Inoculated plates were then incubated at room temperature in a darkened incubator.

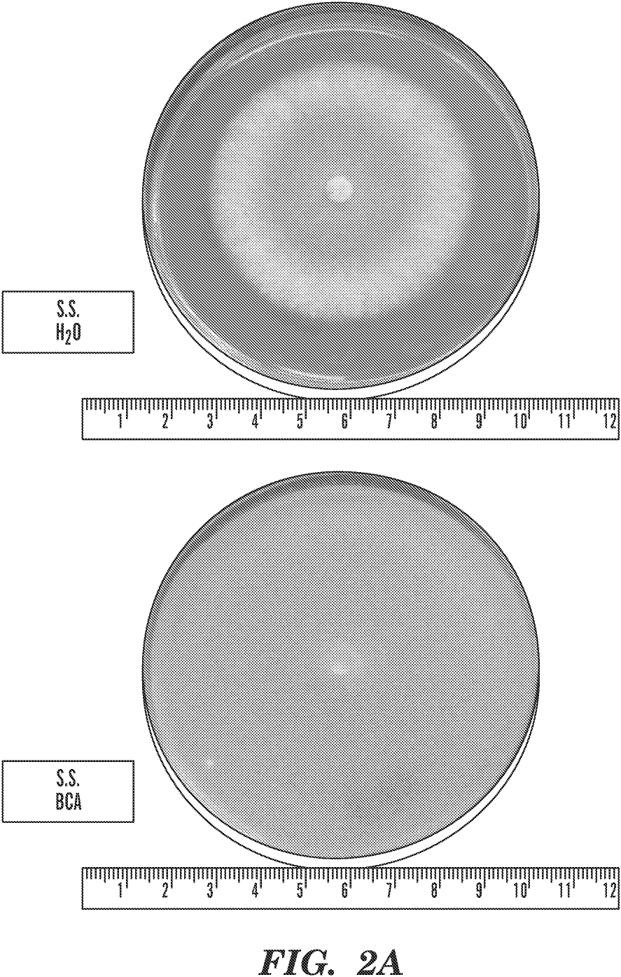

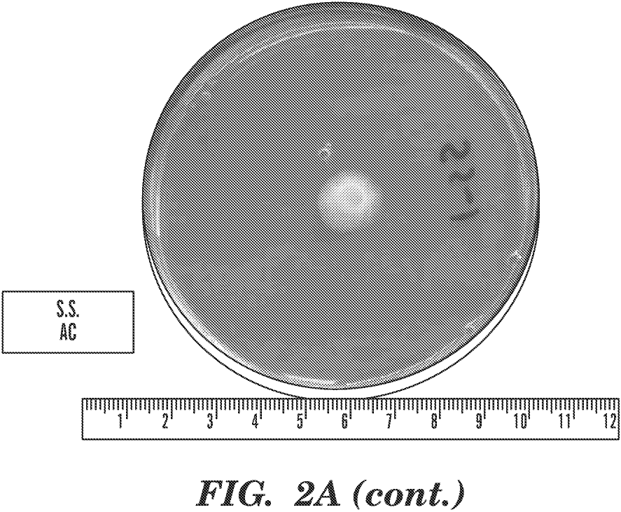

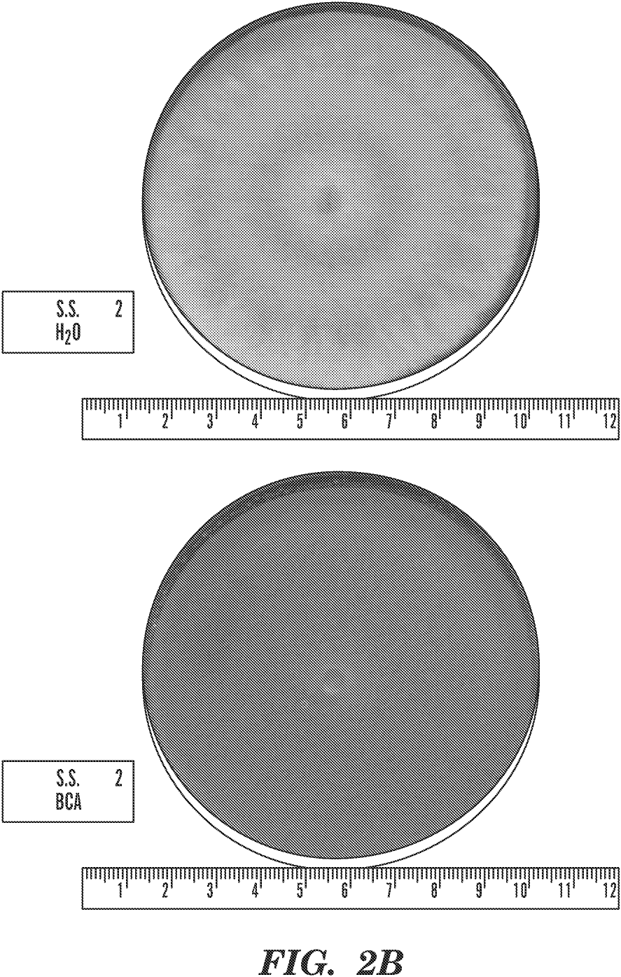

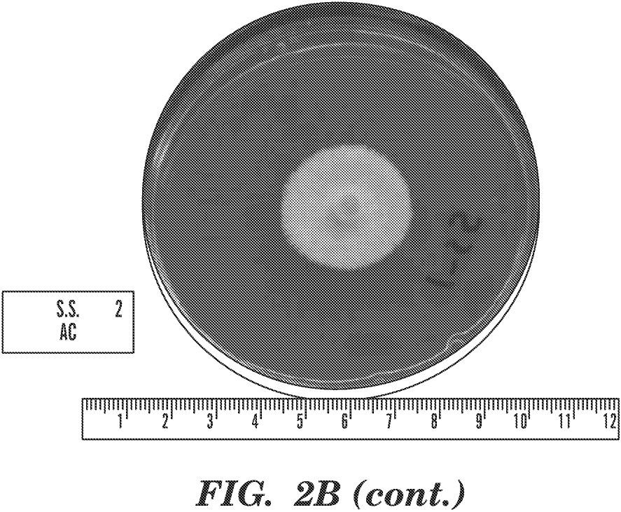

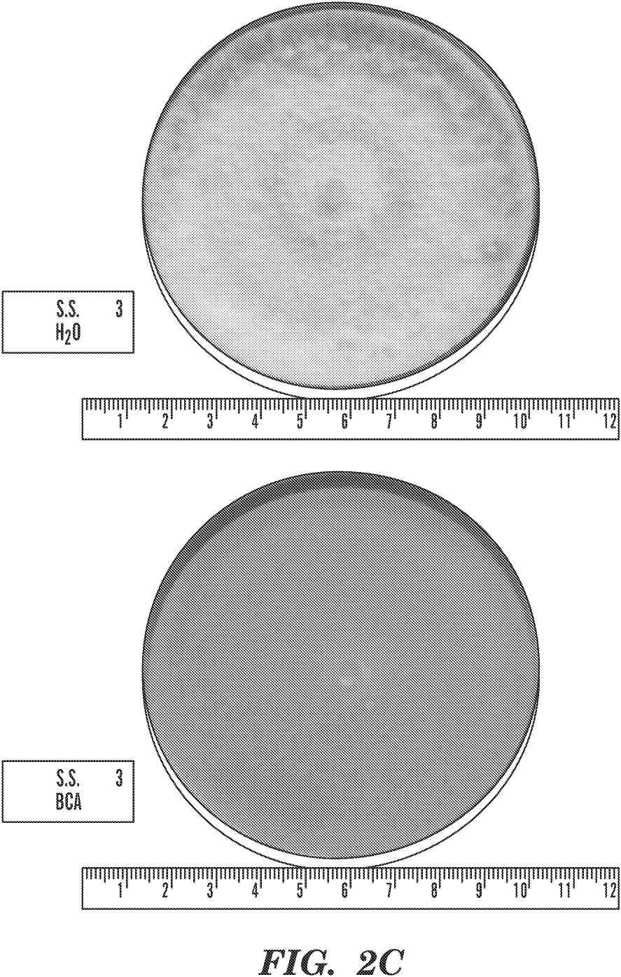

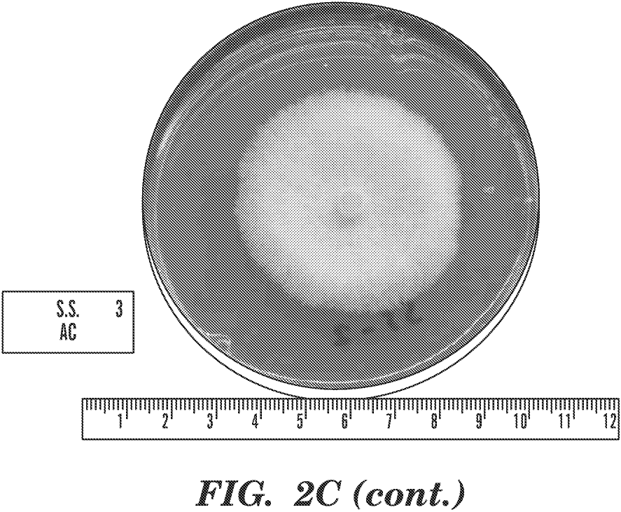

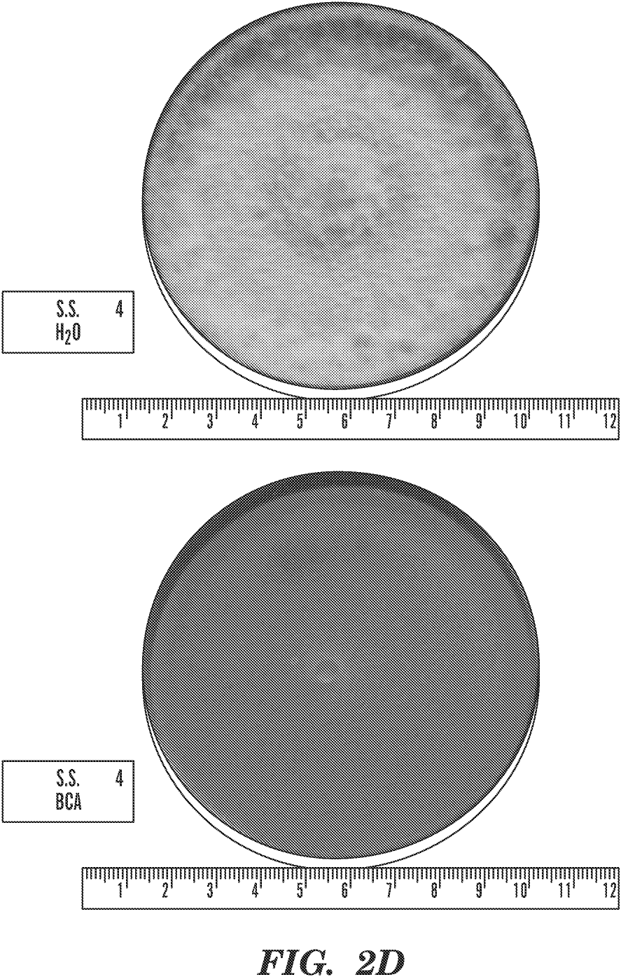

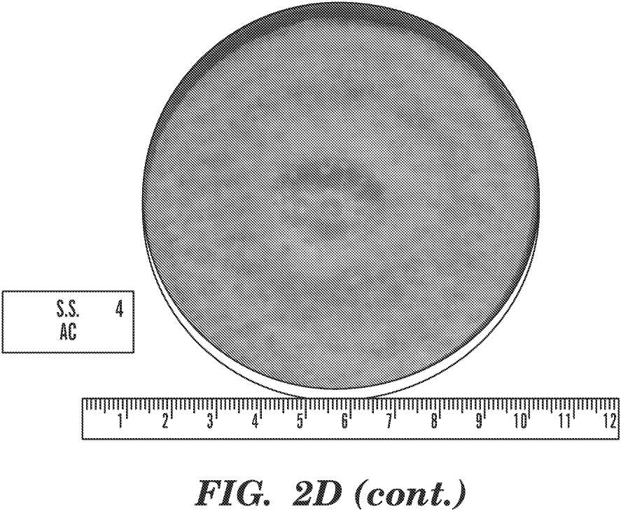

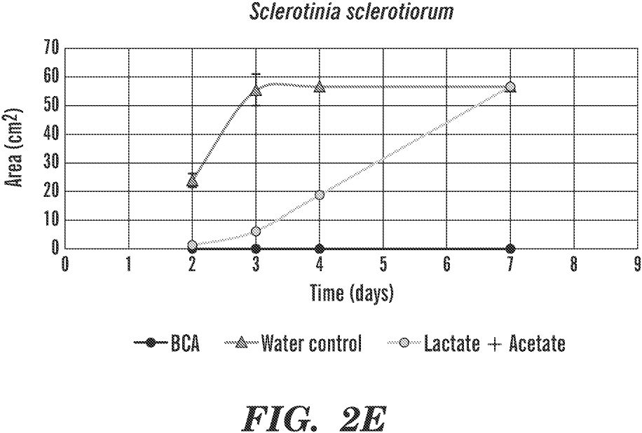

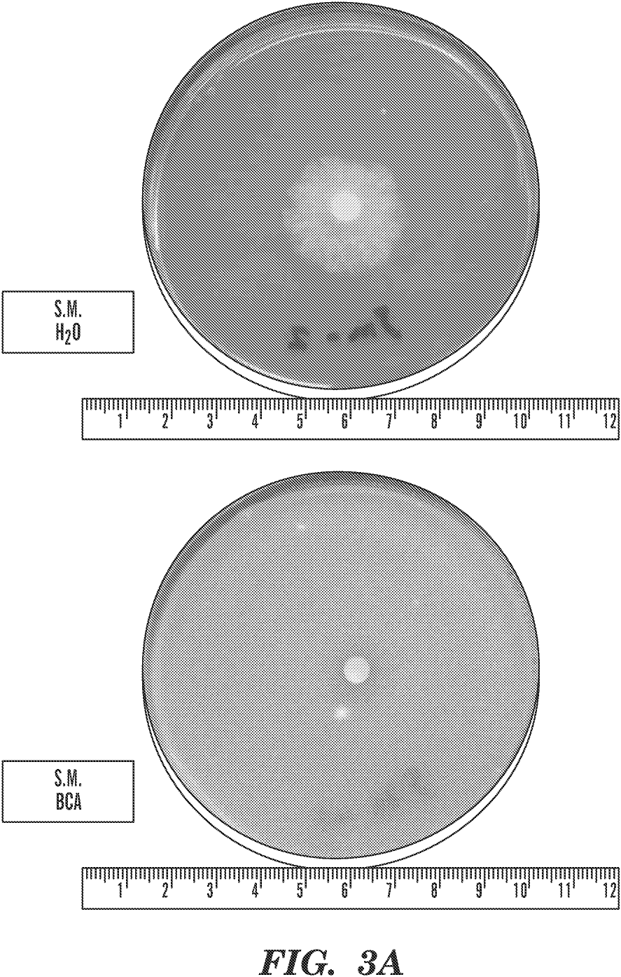

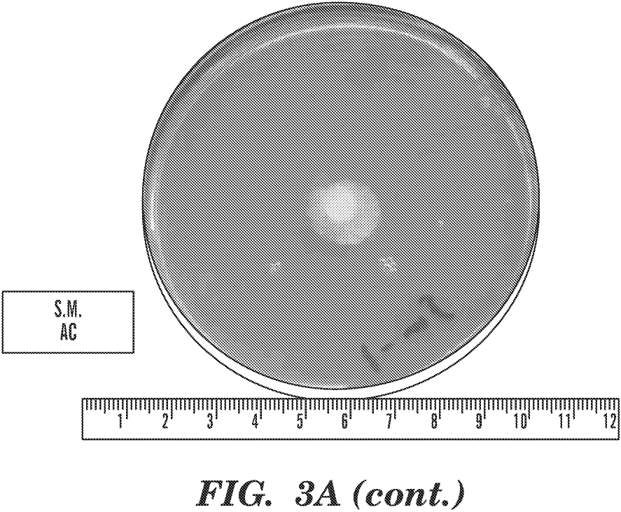

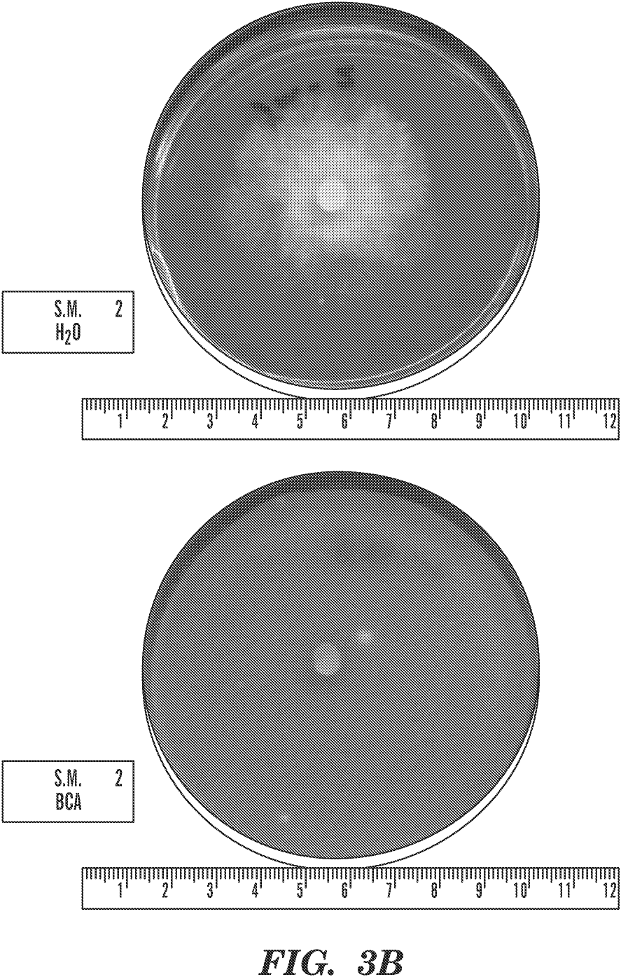

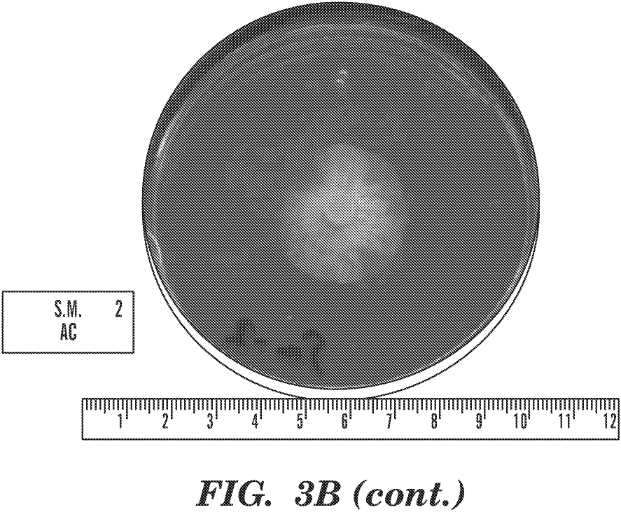

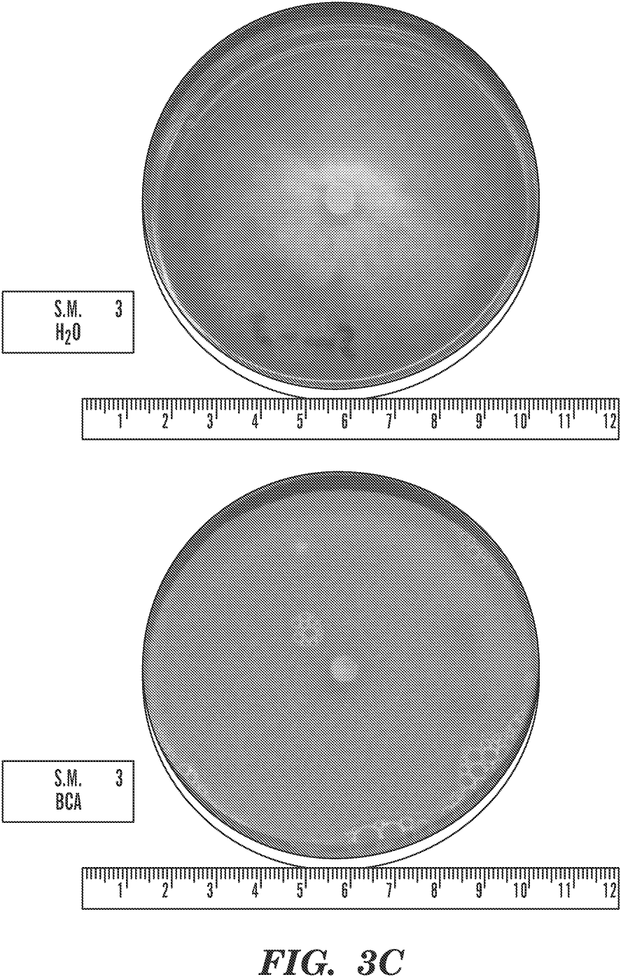

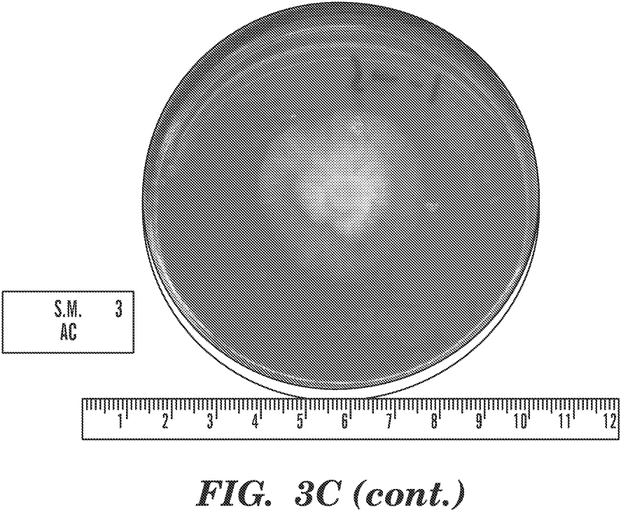

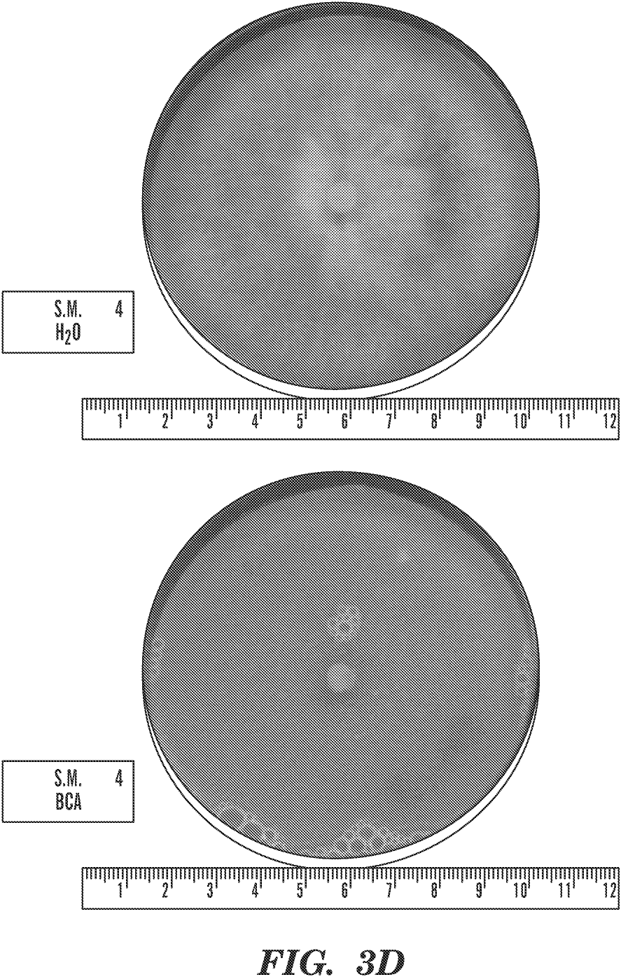

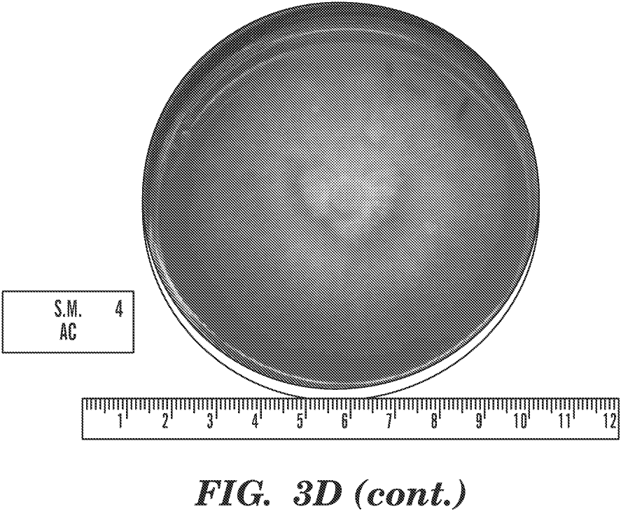

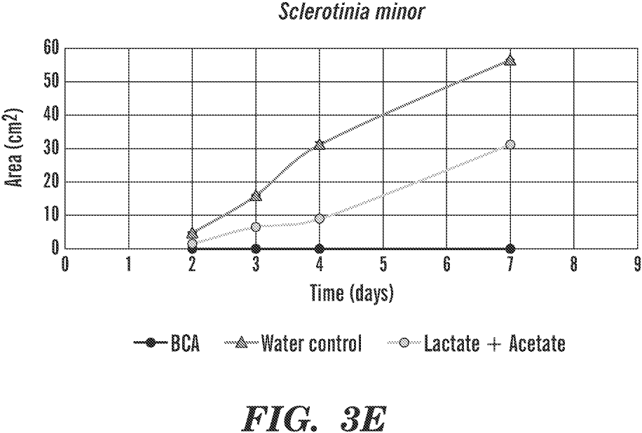

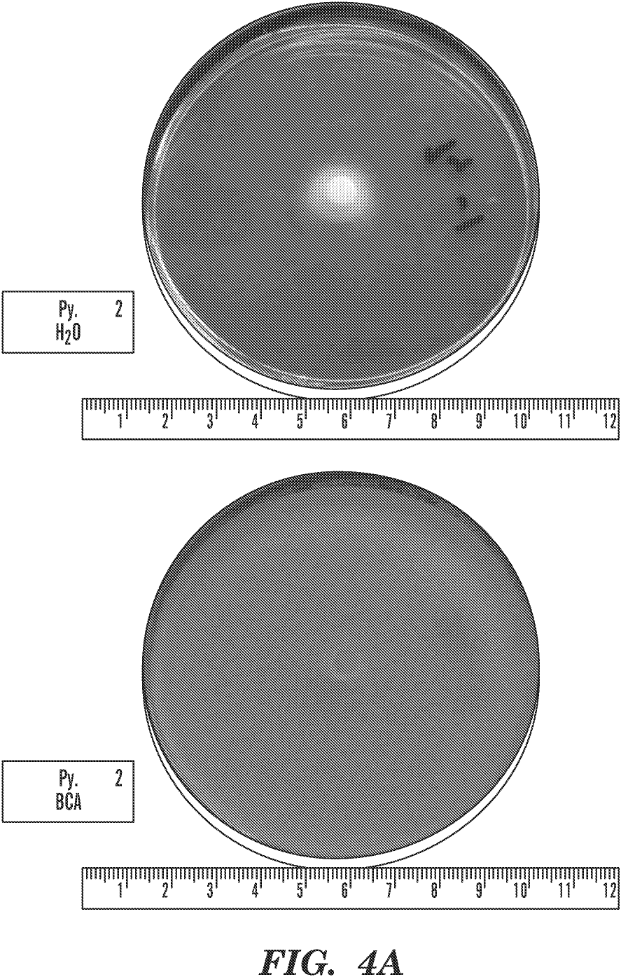

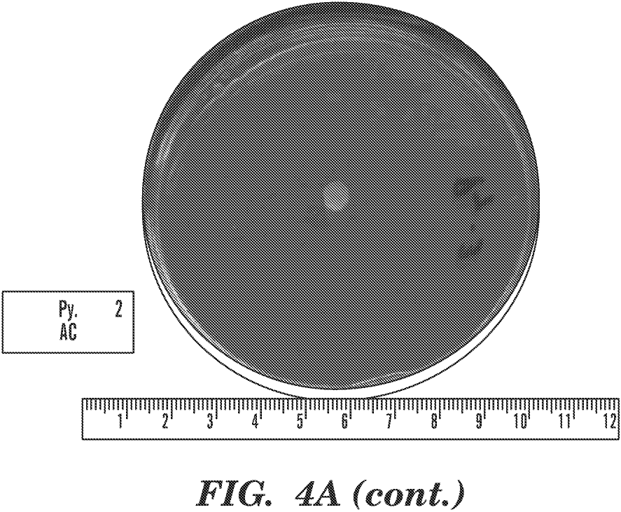

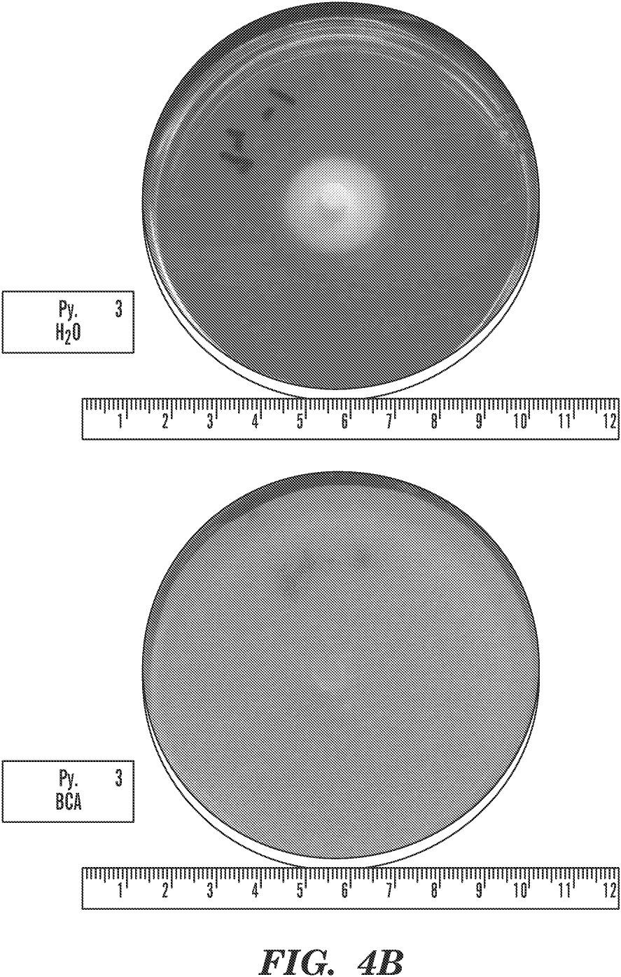

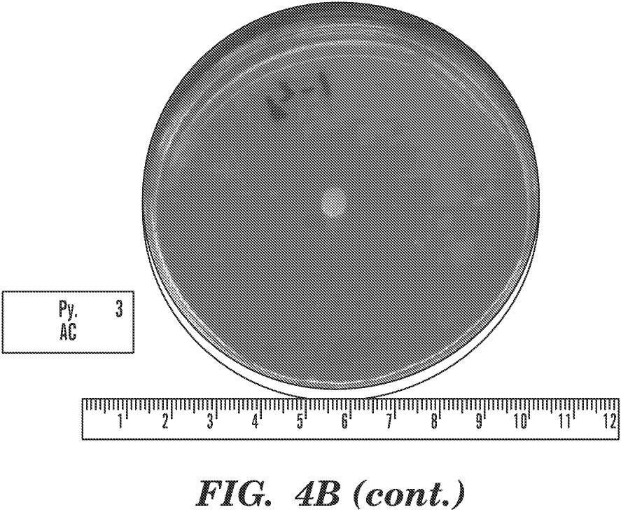

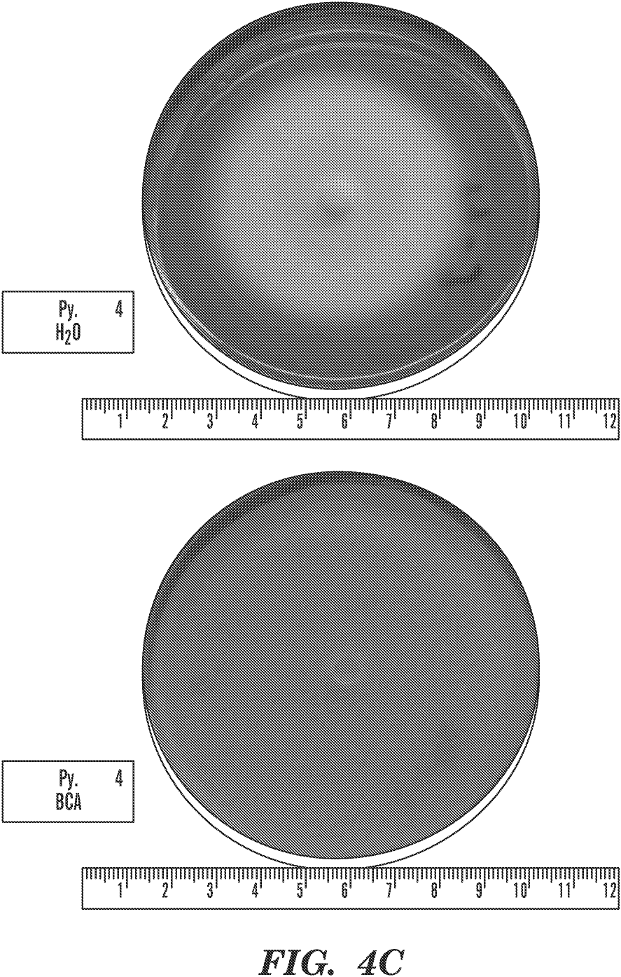

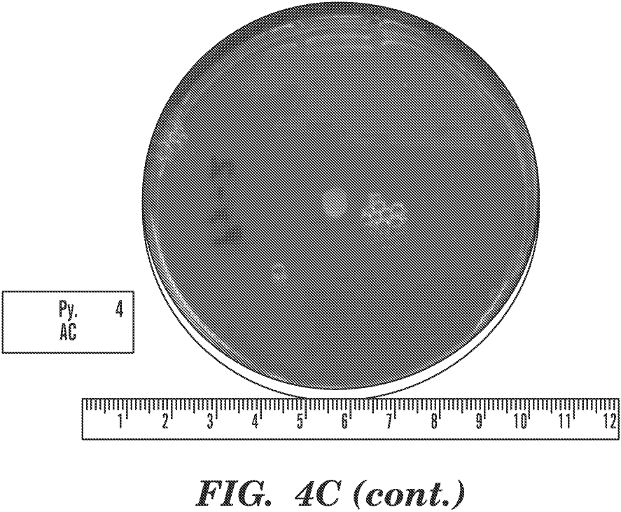

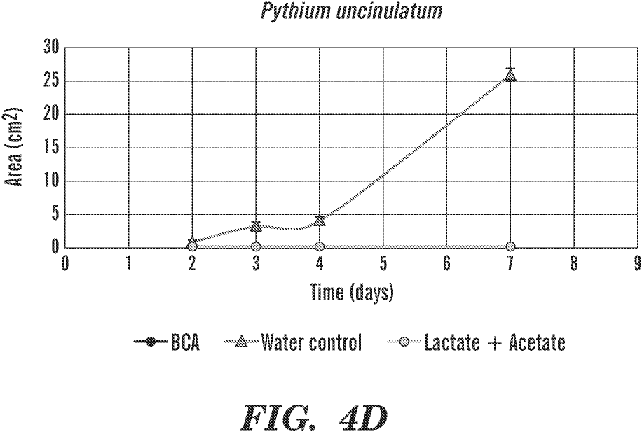

Data on fungal growth were recorded on days 2, 3, 4, and 7. Photographs were taken of the fungal colonies (FIGS. 2A-2D, 3A-3D, and 4A-4C). Area of the fungal growth was determined by analyzing the photos using ImageJ software (FIGS. 2E, 3E, and 4D).

As expected, growth of Scleratinia sclerotiorum and Sclerolinia minor on the negative control plates (H2O) was rapid and reached the maximum area (total area of the petri plate) by day 4. Growth rates were reduced on plates containing the Acetate + L-Lactic acid composition (Lactate + Acetate). No growth occurred on the plates containing the biocontrol agent (BCA).

For Pythium uncinulatum, no growth occurred on any day on the Lactate + Acetate plates. Also, no growth occurred on the plates containing the biocontrol agent (BCA).

The composition containing acetate and L-lactic acid was capable of controlling growth of all three fungal pathogens evaluated.

### Example 3: In Vitro Testing of Compositions Containing Acetate and L-Lactic Acid Against Eight Plant Pathogens

The efficacy of the biocontrol agent in controlling the growth of the agriculturally important fungi including Botrytis cinerea, Colletotrichum acutatum, Fusarium oxysporum f. sp. fragariae, Macrophomina phaseolina, Phytophthora cactorum, Rhizoctonia solani, Sclerotium cepivorum, and Verticillium dahliae was evaluated. Fusarium oxysporum f. sp. fragariae is specialized and causes fusarium wilt of only strawberry. Sclerotium cepivorum also has a narrow host range and causes white rot of Allium crops. The following four fungi were isolated from infected strawberries: Colletotrichum acutatum, Fusarium oxysporum f. sp. fragariae, Macrophomina phaseolina, and Phytophthora cactorum.

Potato dextrose agar plates were prepared, inoculated with each fungal strain, and incubated according to the methods described in Example 2.

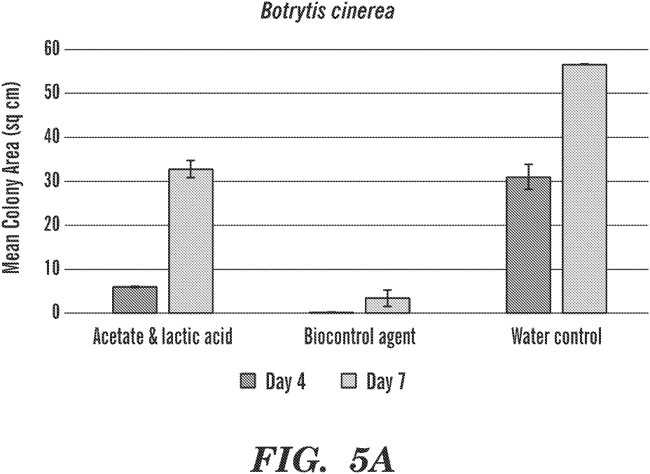

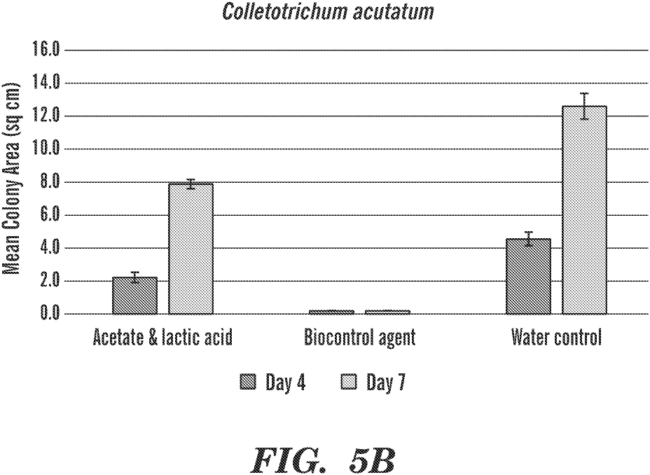

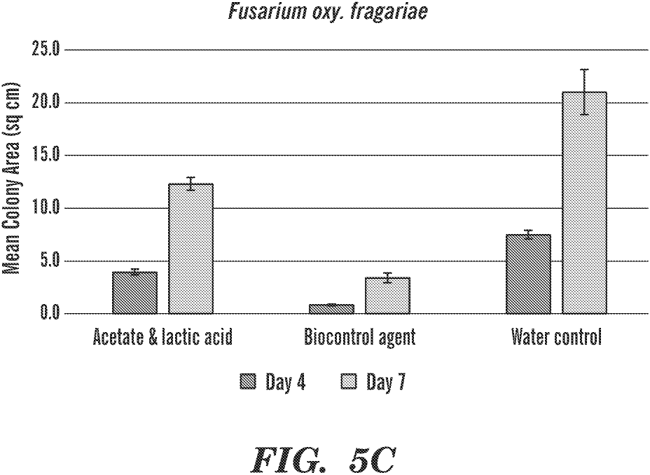

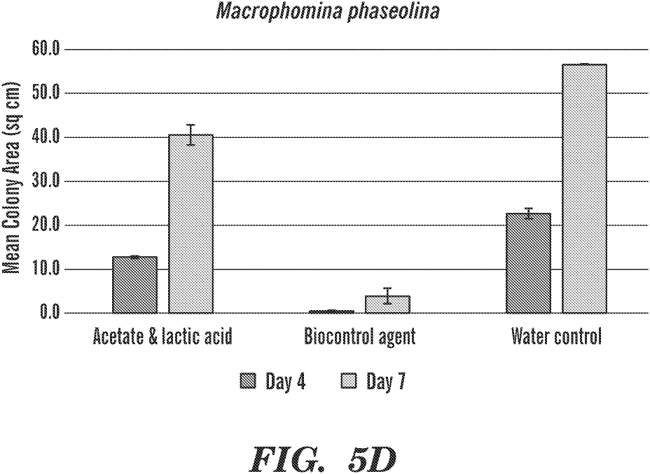

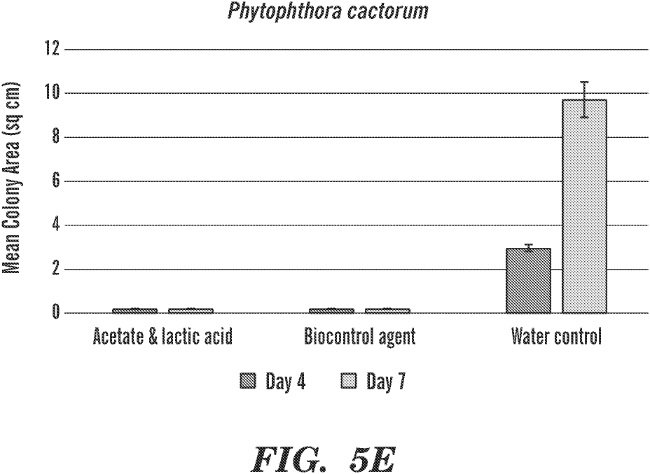

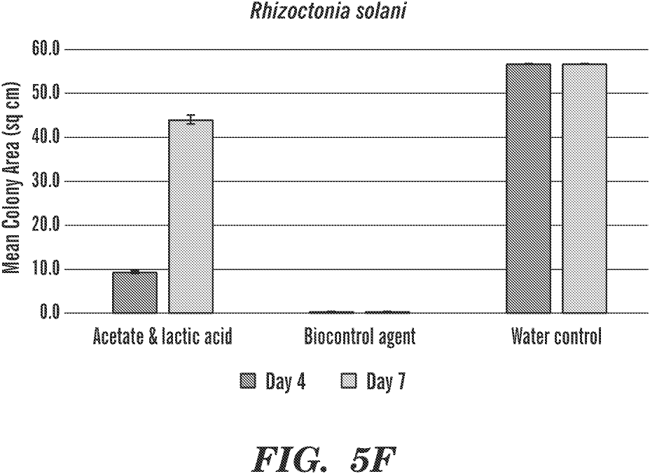

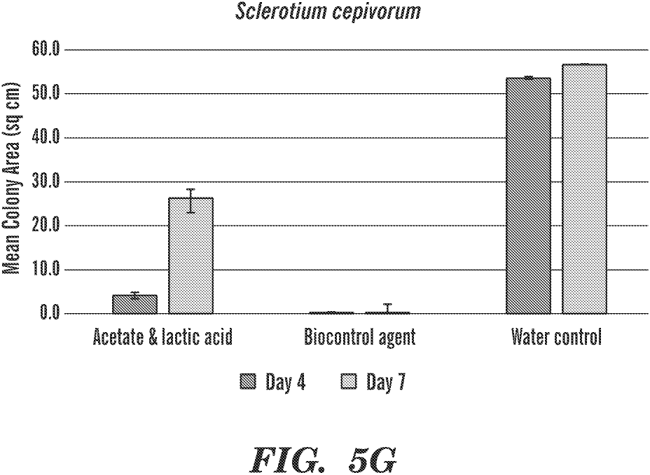

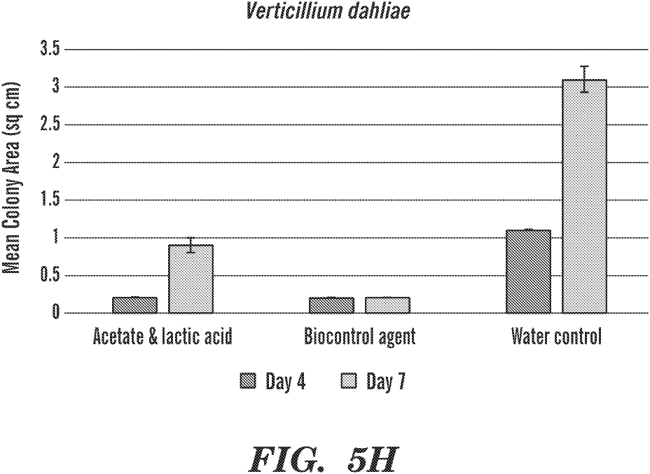

Data on fungal growth were recorded on days 4 and 7 following inoculation. Photographs were taken of the fungal colonies. Area of the fungal growth was determined by analyzing the photos using ImageJ software (FIGS. 5A-5H).

All species grew as expected on the water control Strep-PDA plates. By day 7, fast growing species had covered the entire plate (=56.7 sq cm): Botrytis, Macrophomina, Rhizoctonia, Sclerotium. Moderately fast growing species were Colletotrichum (12.6 sq cm), Fusarium (21.0 sq cm), and Phytophthora (9.7 sq cm). Verticillium is a slow growing fungus and by day 7 reached 3.1 sq cm (Table 2).

By day 7, the composition containing acetate and L-lactate (Acetate & lactic acid) completely inhibited growth of Phytophthora (=0.2 sq cm, the area of the original agar plug) and reduced the growth of Verticillium by 71% (0.9 vs. 3.1 sq cm). The final concentration of each of acetate and lactic acid in the PDA plates were 800 ppm, respectively. Sclerotium cepivorum growth was reduced 54% (263 vs. 56.7 sq cm). For the remaining species, the composition containing acetate and L-lactic acid resulted in growth reductions from 22 to 42%.

Growth of Colletotrichum, Phytophthora, Rhizoctonia, Sclerotium, and Verticillium was completely inhibited by the biocontrol agent. Botrytis (3.4 sq cm), Fusarium (3.4 sq cm), and Macrophomina (3.9 sq cm) each showed very little growth in the presence of the biocontrol agent.

The composition containing acetate and L-lactic acid was capable of controlling growth of all eight fungal pathogens evaluated.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application may be related in subject matter to the inventions described in U.S. Provisional Application No. 62/992,364, the disclosure of which is incorporated herein by reference in its entirety for all purposes. All patents and publications mentioned in this specification are herein incorporated by reference to the same extent as if each independent patent and publication was specifically and individually indicated to be incorporated by reference.

1. A composition comprising levorotatory lactate (L-Lactate) and acetate, wherein the composition is substantially free of dextrorotatory lactate (D-Lactate).

2. The composition of claim 1, wherein the amount of each of L-lactate and acetate is effective to inhibit the growth or survival of a fungal pathogen contacted with the composition.

3. (canceled)

4. The composition of claim 1, wherein the composition comprises from about 50 ppm to about 1,600 ppm L-lactate and from about 50 ppm to about 1,600 ppm acetate.

5-6. (canceled)

7. A method for reducing or eliminating growth of a fungus, the method comprising contacting the fungus with L-lactate and acetate, thereby reducing or eliminating growth of the fungus.

8. A method for inhibiting fungal disease in or on a plant, the method comprising contacting the fungus with L-lactate and acetate, thereby inhibiting the fungal disease.

9. A method for inhibiting white rot on an Allium plant and/or in an Allium growth medium, the method comprising contacting the Allium plant and/or the growth medium with L-lactate and acetate, thereby inhibiting white rot on the Allium plant and/or in the Allium growth medium.

10. A method for inhibiting gray mold in a plant or growth medium, the method comprising contacting a surface of the plant and/or the growth medium with L-lactate and acetate, thereby inhibiting gray mold.

11-12. (canceled)

13. The method of claim 7, wherein the fungus belongs to a genus selected from the group consisting of Botrytis, Colletotrichum, Fusarium, Macrophomina, Phytophthora, Pythium, Rhizoctonia, Sclerotinia, Sclerotiniaceae, Sclerotium, and Verticillium.

14. The method of claim 13, wherein the fungus is Botrytis cinerea.

15. The method of claim 13, wherein the fungus is Colletotrichum acutatum.

16. The method of claim 13, wherein the fungus is Fusarium oxysporum f. sp. fragariae.

17. The method of claim 13, wherein the fungus is Macrophomina phaseolina.

18. The method of claim 13, wherein the fungus is Phytophthora cactorum.

19. The method of claim 13, wherein the fungus is Pythium uncinulatum.

20. The method of claim 13, wherein the fungus is Rhizoctonia solani.

21. The method of claim 13, wherein the fungus is Sclerotinia minor.

22. The method of claim 13, wherein the fungus is Sclerotium cepivorum.

23. The method of claim 13, wherein the fungus is Sclerotinia sclerotiorum.

24. The method of claim 13, wherein the fungus is Verticillium dahliae.

25-26. (canceled)

27. A method for preparing a soil or growth medium for growing an Allium plant, the method comprising contacting the soil or growth medium with a composition comprising L-lactate and acetate, thereby preparing the soil or growth medium.

28. A method for protecting plant surfaces while growing the plant, the method comprising contacting the plant surfaces with a composition comprising L-lactate and acetate, thereby protecting the plant surfaces.

29-39. (canceled)

40. A plant growth medium comprising L-lactate and acetate, wherein the plant growth medium is substantially free of dextrorotatory lactate (D-Lactate).

41. (canceled)
